(19) United States
(12) Patent Application Publication
(10) Pub. No.: US 2009/0318402 A1
(43) Pub. Date: Dec. 24, 2009

(54) RUTHENIUM (II) COMPOUNDS

(75) Inventors: Sarah Dougan, Edinburgh (GB); Abraha Habtemariam, Edinburgh (GB); Michael Melchart, Edinburgh (GB); Peter John Sadler, Edinburgh (GB)

Correspondence Address: CURATOLO SIDOTI CO., LPA, 24500 CENTER RIDGE ROAD, SUITE 280, CLEVELAND, OH, 44145, US

(73) Assignee: The University Court of the University of Edinburg, Edinburgh (GB)

(21) Appl. No.: 12/281,801

(22) PCT Filed: Mar. 7, 2007
(86) PCT No.: PCT/GB2007/000784

§ 371 Date: Jan. 23, 2009

(30) Foreign Application Priority Data
Mar. 7, 2006 (GB) 0604602.3

(51) Int. Cl. A61K 31/555; C07F 15/00; A61P 35/00

(52) U.S. Cl. 514/186; 546/10; 548/108

(57) ABSTRACT

A ruthenium (II) compound of formula (I) wherein X is halo or a neutral or negatively charged O, N- or S-donor ligand; Y is a counterion; m is 0 or 1; q is 1, 2 or 3; A is either: (i) (^{Ru)}—NR^{N1}R^{N2}—R^{N3}—^{(N)}, where R^{N1} and R^{N2} are independently selected from H, optionally substituted C₁₋₇alkyl, C₃₋₂₀ heterocyclyl and C₅₋₂₀aryl, and R^{N3} is C₁₋₂alkylene; or (ii) a nitrogen-containing C₅₋₆aromatic ring, wherein the nitrogen ring atom is bound to the ruthenium atom, and the ring is also bound to the azo-nitrogen, either by a single bond wherein the bond is α or β to the nitrogen ring atom, or by a —CH₂⁻ group wherein the bond is x to the nitrogen ring atom; B is optionally substituted C₁₋₇alkyl, C₃₋₂₀heterocyclyl or C₅₋₂₀aryl.

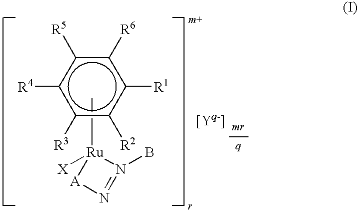

This invention relates to ruthenium (II) compounds, to their use in medicine, particularly for the treatment and/or prevention of cancer, and to a process for their preparation.

WO 01/30790, WO 02/02572, WO 2004/005304 and WO 2004/096819 disclose ruthenium (II) compounds for use in the treatment of cancer. These compounds can be described as half-sandwich compounds, having an arene ring bound to the ruthenium, as well as other non-arene ligands. The compounds exemplified in these applications have as one of the ligands a halo atom. It is thought that the hydrolysis of the halo atom activates the complexes and allows them to bind to DNA. More recently it has been found that complexes containing ligands that have longer hydrolysis times still exhibit anti-tumour activity (Sadler et al, Proc. Natl. Acad. Sci. USA, 2005, 102, 18269).

The present inventors have discovered that a new class of ruthenium (II) sandwich complexes also show anti-tumour activity.

According to a first aspect of the present invention there is provided a ruthenium (II) compound of formula (I):

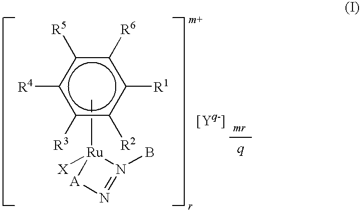

or a solvate or prodrug thereof, wherein:

R¹, R², R³, R⁴, R⁵ and R⁶ are independently selected from H, C₁₋₇ alkyl, C₅₋₂₀ aryl, C₃₋₂₀ heterocyclyl, halo, ester, amido, acyl, sulfo, sulfonamido, ether, thioether, azo, amino, or R¹ and R² together with the ring to which they are attached form a saturated or unsaturated carbocyclic or heterocyclic group containing up to three 3- to 8-membered carbocyclic or heterocyclic rings, wherein each carbocyclic or heterocyclic ring may be fused to one or more other-carbocyclic or heterocyclic rings;

X is halo or a neutral or negatively charged O, N- or S-donor ligand;

Y is a counterion;

m is 0 or 1;

q is 1, 2 or 3;

A is either:
(i) ^{(Ru)}—NR^{N1}R^{N2}—R^{N3}—^{(N)}, where R^{N1} and R^{N2} are independently selected from H, optionally substituted C₁₋₇ alkyl, C₃₋₂₀ heterocyclyl and C₅₋₂₀ aryl, and R^{N3} is C₁₋₂ alkylene; or
(ii) a nitrogen-containing C₅₋₆ aromatic ring, wherein the nitrogen ring atom is bound to the ruthenium atom, and the ring is also bound to the azo-nitrogen, either by a single bond wherein the bond is α or β to the nitrogen ring atom, or by a —CH₂— group wherein the bond is a to the nitrogen ring atom;

B is optionally substituted C₁₋₇ alkyl, C₃₋₂₀ heterocyclyl or C₅₋₂₀ aryl;

the compound of formula (I) optionally being in the form of a dimer in which:

(a) the B group from each moiety are linked through a linking group which is a single bond, —O—, —NH—, C₁₋₆ alkylene or C₅₋₂₀ arylene; or

(b) one group serves as the B group for both moieties, i.e. C₁₋₇ alkylene, C₃₋₂₀ heterocyclylene or C₅₋₂₀ arylene; or

(c) R¹ on each moiety together form a linking group which is a single bond, —O—, C₁₋₆ alkylene or C₅₋₂₀ arylene.

For illustration purposes, some examples of the types of complex provided by case (ii) above, wherein A is pyridine, are shown in the table below:

| | single bond | —CH₂— |
|---|---|---|
| α | 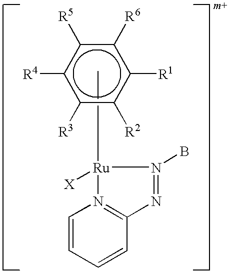 | 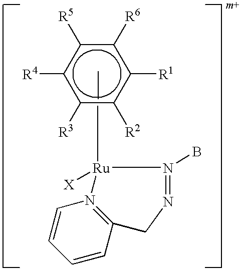 |
| β | 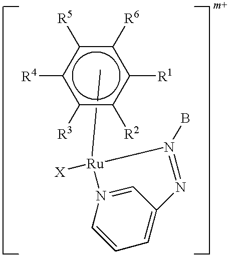 | — |

Without wishing to be bound by any theory, the solution chemistry of these complexes is very different to those previously disclosed as being active for use in treating cancer, as in most cases the group X does not readily hydrolyze. It is therefore thought that the present complexes may have a different mode of action, in which the intact complex is the active species.

A second aspect of the present invention provides a composition comprising a compound of the first aspect and a pharmaceutically acceptable carrier or diluent.

A third aspect of the invention provides the use of a compound of the first aspect in a method of therapy.

A fourth aspect of the invention provides the use of a compound of the first aspect in the preparation of a medicament for the treatment of cancer.

A fifth aspect of the invention provides a method of treatment of a subject suffering from cancer, comprising administering to such a subject a therapeutically-effective amount of a compound of the first aspect, preferably in the form of a pharmaceutical composition.

Definitions

N-donor ligands: N-donor ligands are ligands which bind to a metal atom via a nitrogen atom. They are well known in the art and include: nitrile ligands (N≡C—R); azo ligands (N═N—R); aromatic N-donor ligands; amine ligands (NR^{N4}R^{N5}R^{N6}); azide (N₃⁻); cyanide (N≡C⁻); isothiocyanate (NCS⁻).

In both nitrile and azo ligands R may be selected from C₁₋₇ alkyl and C₅₋₂₀ aryl.

Aromatic N-donor ligands include optionally substituted pyridine, pyridazine, pyrimidine, purine and pyrazine. The optional substituents may be selected from cyano, halo and C₁₋₇ alkyl.

R^{N4}, R^{N5} and R^{N6} may be independently selected from H and C₁₋₇ alkyl.

S-donor ligands: S-donor ligands are ligands which bind to a metal atom via a sulphur atom. They are well known in the art and include: thiosulfate (S₂O₃²⁻); isothiocyanate (NCS⁻); thiocyanate (CNS⁻); sulfoxide ligands (R^{S1}R^{S2}SO); thioether ligands (R^{S1}R^{S2}S); thiolate ligands (R^{S1}S⁻); sulfinate ligands (R^{S1}SO₂⁻); and sulfenate ligands (R^{S1}SO⁻), wherein R^{S1} and R^{S2} are independently selected from C₁₋₇ alkyl and C₅₋₂₀ aryl, which groups may be optionally substituted.

O-donor ligands: O-donor ligands are ligands which bind to a metal atom via an oxygen atom. They are well known in the art and include: water (H₂O), carbonate (CO₃⁻); carboxylate ligands (R^{C}CO₂⁻); nitrate (NO₃⁻); sulfate (SO₄²⁻) and sulphonate (R^{S1}O₃⁻), wherein R^{C} is selected from C₁₋₇ alkyl and C₅₋₂₀ aryl and R^{S1} is as defined above.

C₁₋₇ Alkyl: The term “C₁₋₇ alkyl”, as used herein, pertains to a monovalent moiety obtained by removing a hydrogen atom from a carbon atom of a hydrocarbon compound having from 1 to 7 carbon atoms, which may be aliphatic or alicyclic, and which may be saturated or unsaturated (e.g., partially unsaturated, fully unsaturated). Thus, the term “alkyl” includes the sub-classes alkenyl, alkynyl, cycloalkyl, cycloalkyenyl, cylcoalkynyl, etc., discussed below.

Examples of saturated C₁₋₇ alkyl groups include, but are not limited to, methyl (C₁), ethyl (C₂), propyl (C₃), butyl (C₄), pentyl (C₅), hexyl (C₆) and heptyl (C₇).

Examples of saturated linear C₁₋₇ alkyl groups include, but are not limited to, methyl (C₁), ethyl (C₂), n-propyl (C₃), n-butyl (C₄), n-pentyl (amyl) (C₅), n-hexyl (C₆), and n-heptyl (C₇).

Examples of saturated branched C₁₋₇ alkyl groups include iso-propyl (C₃), iso-butyl (C₄), sec-butyl (C₄), tert-butyl (C₄), iso-pentyl (C₅), and neo-pentyl (C₅).

C₂₋₇ Alkenyl: The term “C₂₋₇ alkenyl”, as used herein, pertains to an alkyl group having one or more carbon-carbon double bonds. Examples of C₂₋₇ alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂), butenyl (C₄), pentenyl (C₅), and hexenyl (C₆).

C₂₋₇ Alkynyl: The term “C₂₋₇ alkynyl”, as used herein, pertains to an alkyl group having one or more carbon-carbon triple bonds. Examples of C₂₋₇ alkynyl groups include, but are not limited to, ethynyl (ethinyl, —C≡CH) and 2-propynyl (propargyl, —CH₂—C≡CH).

C₃₋₇ Cycloalkyl: The term “C₃₋₇ cycloalkyl”, as used herein, pertains to an alkyl group which is also a cyclyl group; that is, a monovalent moiety obtained by removing a hydrogen atom from an alicyclic ring atom of a carbocyclic ring of a carbocyclic compound, which carbocyclic ring may be saturated or unsaturated (e.g., partially unsaturated, fully unsaturated), which moiety has from 3 to 7 carbon atoms. Thus, the term “C₃₋₇ cycloalkyl” includes the sub-classes cycloalkyenyl and cycloalkynyl. Examples of cycloalkyl groups include, but are not limited to, those derived from:
saturated hydrocarbon compounds:

cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇), methylcyclohexane (C₇); and
unsaturated hydrocarbon compounds:

cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇).

The alkyl groups in the compounds of the invention may optionally be substituted. Substituents include one or more further alkyl groups and/or one or more further substituents, such as, for example, C₅₋₂₀ aryl (e.g. benzyl), C₃₋₂₀ heterocyclyl, amino, cyano (—CN), nitro (—NO₂), hydroxyl (—OH), ester, halo, thiol (—SH), thioether and sulfonate (—S(═O)₂)OR, where R is wherein R is a sulfonate substituent, for example, a C₁₋₇ alkyl group, a C₃₋₂₀ heterocyclyl group, or a C₅₋₂₀ aryl group, preferably a C₁₋₇ alkyl group).

C₁₋₁₂ alkylene: The term “C₁₋₁₂ alkylene” is defined similarly to the definition of the term “alkyl” and is a divalent species obtained by removing two hydrogen atoms from a carbon atom of a hydrocarbon compound having from 1 to 12 carbon atoms, which may be aliphatic or alicyclic, and which may be saturated or unsaturated (e.g., partially unsaturated, fully unsaturated). The radicals may be separated by one or more carbon atoms linked in a chain, except in the case of C₁ alkylene where the radicals are on the same carbon atom (i.e. a —CH₂— group). Preferably, the alkylene groups are straight chain groups. C₁₋₁₂ alkylene groups are optionally substituted in the alkylene chain.

C₃₋₂₀ Heterocyclyl: The term “C₃₋₂₀ heterocyclyl”, as used herein, pertains to a monovalent moiety obtained by removing a hydrogen atom from a ring atom of a heterocyclic compound, which moiety has from 3 to 20 ring atoms, of which from 1 to 10 are ring heteroatoms. Preferably, each ring has from 3 to 7 ring atoms, of which from 1 to 4 are ring heteroatoms.

In this context, the prefixes (e.g., C₃₋₂₀, C₃₋₇, C₅₋₆, etc.) denote the number of ring atoms, or range of number of ring atoms, whether carbon atoms or heteroatoms. For example, the term “C₅₋₆heterocyclyl”, as used herein, pertains to a heterocyclyl group having 5 or 6 ring atoms. Examples of groups of heterocyclyl groups include C₃₋₂₀ heterocyclyl, C₅₋₂₀ heterocyclyl, C₅₋₂₀ heteroaryl, C₃₋₁₅ heterocyclyl, C₅₋₁₅ heterocyclyl, C₃₋₁₂ heterocyclyl, C₅₋₁₂ heterocyclyl, C₃₋₁₀ heterocyclyl, C₅₋₁₀ heterocyclyl, C₃₋₇ heterocyclyl, C₅₋₇ heterocyclyl, and C₅₋₆ heterocyclyl.

Examples of monocyclic heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);

O₁: oxirane (C₃), oxetane (C₄), oxolane (tetrahydrofuran) (C₅), oxole (dihydrofuran) (C₅), oxane (tetrahydropyran) (C₆), dihydropyran, (C₆), pyran (C₆), oxepin (C₇);

S₁: thiirane (C₃), thietane (C₄), thiolane (tetrahydrothiophene) (C₅), thiane (tetrahydrothiopyran) (C₆), thiepane (C₇);

O₂: dioxolane (C₅), dioxane (C₆), and dioxepane (C₇);

O₃: trioxane (C₆);

N₂: imidazolidine (C₅), pyrazolidine (diazolidine) (C₅), imidazoline (C₅), pyrazoline (dihydropyrazole) (C₅), piperazine (C₆);

N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);

N₁S₁: thiazoline (C₅), thiazolidine (C₅), thiomorpholine (C₆);

N₂O₁: oxadiazine (C₆);

O₁S₁: oxathiole (C₅) and oxathiane (thioxane) (C₆); and

N₁O₁S₁: oxathiazine (C₆).

C₃₋₂₀ heterocyclyl groups may optionally be substituted with one or more substituents including, for example, C₁₋₇ alkyl, C₅₋₂₀ aryl, C₃₋₂₀ heterocyclyl, amino, cyano, nitro, hydroxyl, ester, halo, thiol, thioether and sulfonate.

C₃₋₂₀ heterocyclylene: The term “C₃₋₂₀ heterocyclylene” is defined similarly to the definition of the term “heterocyclyl” and is a divalent species obtained by removing two hydrogen atoms from ring atoms of an heterocyclic compound, which moiety has from 3 to 20 ring atoms. The radicals may be separated by one or more ring atoms, and may be in different rings.

C₅₋₂₀ Aryl: The term “C₅₋₂₀ aryl”, as used herein, pertains to a monovalent moiety obtained by removing a hydrogen atom from an aromatic ring atom of an aromatic compound, which moiety has from 3 to 20 ring atoms. Preferably, each ring has from 5 to 7 ring atoms.

In this context, the prefixes (e.g., C₃₋₂₀, C₅₋₇, C₅₋₆, etc.) denote the number of ring atoms, or range of number of ring atoms, whether carbon atoms or heteroatoms. For example, the term “C₅₋₆ aryl”, as used herein, pertains to an aryl group having 5 or 6 ring atoms.

The ring atoms may be all carbon atoms, as in “carboaryl groups”. Examples of carboaryl groups include C₃₋₂₀ carboaryl, C₅₋₂₀ carboaryl, C₅₋₁₅ carboaryl, C₅₋₁₂ carboaryl, C₅₋₁₀ carboaryl, C₅₋₇ carboaryl, C₅₋₆ carboaryl, C₅ carboaryl, and C₆ carboaryl.

Examples of carboaryl groups include, but are not limited to, those derived from benzene (i.e., phenyl) (C₆), naphthalene (C₁₀), azulene (C₁₀), anthracene (C₁₄), phenanthrene (C₁₄), naphthacene (C₁₈), and pyrene (C₁₆).

Examples of aryl groups which comprise fused rings, at least one of which is an aromatic ring, include, but are not limited to, groups derived from indane (e.g., 2,3-dihydro-1H-indene) (C₉), indene (C₉), isoindene (C₉), tetraline (1,2,3,4-tetrahydronaphthalene (C₁₀), acenaphthene (C₁₂), fluorene (C₁₃), phenalene (C₁₃), acephenanthrene (C₁₅), and aceanthrene (C₁₆).

Alternatively, the ring atoms may include one or more heteroatoms, as in “heteroaryl groups”. Examples of heteroaryl groups include C₃₋₂₀ heteroaryl, C₅₋₂₀ heteroaryl, C₅₋₁₅ heteroaryl, C₅₋₁₂ heteroaryl, C₅₋₁₀ heteroaryl, C₅₋₇ heteroaryl, C₅₋₆ heteroaryl, C₅ heteroaryl, and C₆ heteroaryl.

Examples of monocyclic heteroaryl groups include, but are not limited to, those derived from:

N₁: pyrrole (azole) (C₅), pyridine (azine) (C₆);

O₁: furan (oxole) (C₅);

S₁: thiophene (thiole) (C₅);

N₁O₁: oxazole (C₅), isoxazole (C₅), isoxazine (C₆);

N₂O₁: oxadiazole (furazan) (C₅);

N₃O₁: oxatriazole (C₅);

N₁S₁: thiazole (C₅), isothiazole (C₅);

N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆);

N₃: triazole (C₅), triazine (C₆); and,

N₄: tetrazole (C₅).

Examples of heteroaryl groups which comprise fused rings, include, but are not limited to:
C₉ heteroaryl groups (with 2 fused rings) derived from benzofuran (O₁), isobenzofuran (O₁), indole (N₁), isoindole (N₁), indolizine (N₁), indoline (N₁), isoindoline (N₁), purine (N₄) (e.g., adenine, guanine), benzimidazole (N₂), indazole (N₂), benzoxazole (N₁O₁), benzisoxazole (N₁O₁), benzodioxole (O₂), benzofurazan (N₂O₁), benzotriazole (N₃), benzothiofuran (S₁), benzothiazole (N₁S₁), benzothiadiazole (N₂S);

C₁₀ heteroaryl groups (with 2 fused rings) derived from chromene (O₁), isochromene (O₁), chroman (O₁), isochroman (O₁), benzodioxan (O₂), quinoline (N₁), isoquinoline (N₁), quinolizine (N₁), benzoxazine (N₁O₁), benzodiazine (N₂), pyridopyridine (N₂), quinoxaline (N₂), quinazoline (N₂), cinnoline (N₂), phthalazine (N₂), naphthyridine (N₂), pteridine (N₄);

C₁₁ heteroaryl groups (with 2 fused rings) derived from benzodiazepine (N₂);

C₁₃ heteroaryl groups (with 3 fused rings) derived from carbazole (N₁), dibenzofuran (O₁), dibenzothiophene (S₁), carboline (N₂), perimidine (N₂), pyridoindole (N₂); and,

C₁₄ heteroaryl groups (with 3 fused rings) derived from acridine (N₁), xanthene (O₁), thioxanthene (S₁), oxanthrene (O₂), phenoxathiin (O₁S₁), phenazine (N₂), phenoxazine (N₁O₁), phenothiazine (N₁S₁), thianthrene (S₂), phenanthridine (N₁), phenanthroline (N₂), phenazine (N₂).

C₅₋₂₀ aryl groups may optionally be substituted with one or more substituents including, for example, C₁₋₇ alkyl, C₅₋₂₀ aryl, C₃₋₂₀ heterocyclyl, amino, cyano, nitro, hydroxyl, ester, halo, thiol, thioether and sulfonate.

C₅₋₂₀ arylene: The term “C₅₋₂₀ arylene” is defined similarly to the definition of the term “aryl” and is a divalent species obtained by removing two hydrogen atoms from an aromatic ring atom of an aromatic compound, which moiety has from 5 to 20 ring atoms. The radicals may be separated by one or more ring atoms, and may be in different rings.

Halo: —F, —Cl, —Br, and —I.

Ester (carboxylate, carboxylic acid ester, oxycarbonyl): —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Examples of ester groups include, but are not limited to, —C(═O)OCH₃, —C(═O)OCH₂CH₃, —C(═O)OC(CH₃)₃, and —C(═O)OPh.

Amino: —NR¹R², wherein R¹ and R² are independently amino substituents, for example, hydrogen, a C₁₋₇ alkyl group (also referred to as C₁₋₇ alkylamino or di-C₁₋₇ alkylamino), a C₃₋₂₀ heterocyclyl group, or a C₅₋₂₀ aryl group, preferably H or a C₁₋₇ alkyl group, or, in the case of a “cyclic” amino group, R¹ and R², taken together with the nitrogen atom to which they are attached, form a heterocyclic ring having from 4 to 8 ring atoms. Amino groups may be primary (—NH₂), secondary (—NHR¹), or tertiary (—NHR¹R²), and in cationic form, may be quaternary (—⁺NR¹R²R³). Examples of amino groups include, but are not limited to, —NH₂, —NHCH₃, —NHC(CH₃)₂, —N(CH₃)₂, —N(CH₂CH₃)₂, —NHCH₂Ph and —NHPh. Examples of cyclic amino groups include, but are not limited to, aziridino, azetidino, pyrrolidino, piperidino, piperazino, morpholino, and thiomorpholino.

Amido (carbamoyl, carbamyl, aminocarbonyl, carboxamide): —C(═O)NR R², wherein R¹ and R² are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinocarbonyl.

Acyl (keto): —C(═O)R, wherein R is an acyl substituent, for example, a C₁₋₇ alkyl group (also referred to as C₁₋₇ alkylacyl or C₁₋₇ alkanoyl), a C₃₋₂₀ heterocyclyl group (also referred to as C₃₋₂₀ heterocyclylacyl), or a C₅₋₂₀ aryl group (also referred to as C₅₋₂₀ arylacyl), preferably a C₁₋₇ alkyl group. Examples of acyl groups include, but are not limited to, —C(═O)CH₃ (acetyl), —C(═O)CH₂CH₃ (propionyl), —C(═O)C(CH₃)₃ (t-butyryl), and —C(═O)Ph (benzoyl, phenone).

Sulfo: —S(═O)₂OH, —SO₃H.

Sulfonamido (sulfinamoyl; sulfonic acid amide; sulfonamide): —S(═O)₂NR¹R², wherein R¹ and R² are independently amino substituents, as defined for amino groups. Examples of sulfonamido groups include, but are not limited to, —S(═O)₂NH₂, —S(═O)₂NH(CH₃), —S(═O)₂N(CH₃)₂, —S(═O)₂NH(CH₂CH₃), —S(═O)₂N(CH₂CH₃)₂, and —S(═O)₂NHPh.

Ether: —OR, wherein R is an ether substituent, for example, a C₁₋₇ alkyl group (also referred to as a C₁₋₇ alkoxy group), a C₃₋₂₀ heterocyclyl group (also referred to as a C₃₋₂₀ heterocyclyloxy group), or a C₅₋₂₀ aryl group (also referred to as a C₅₋₂₀ aryloxy group), preferably a C₁₋₇ alkyl group.

Thioether (sulfide): —SR, wherein R is a thioether substituent, for example, a C₁₋₇ alkyl group (also referred to as a C₁₋₇ alkylthio group), a C₃₋₂₀ heterocyclyl group, or a C₅₋₂₀ aryl group, preferably a C₁₋₇ alkyl group. Examples of C₁₋₇ alkylthio groups include, but are not limited to, —SCH₃ and —SCH₂CH₃.

Azo: —N═N—R, where R is an azo substituent, for example a C₁₋₇ alkyl group, a C₃₋₂₀ heterocyclyl group, or a C₅₋₂₀ aryl group, preferably a C₁₋₇ alkyl group. Examples of azo groups include, but are not limited to, —N═N—CH₃ and —N═N—Ph.

Heterocyclic ring: The term “heterocyclic ring” as used herein refers to a 3-, 4-, 5-, 6-, 7-, or 8- (preferably 5-, 6- or 7-) membered saturated or unsaturated ring, which may be aromatic or non-aromatic, containing from one to three heteroatoms independently selected from N, O and S, e.g. indole (also see above).

Carbocyclic ring: The term “carbocyclic ring” as used herein refers to a saturated or unsaturated ring, which may be aromatic or non-aromatic, containing from 3 to 8 carbon atoms (preferably 5 to 7 carbon atoms) and includes, for example, cyclopropane, cyclobutane, cyclopentane, cyclohexane and cycloheptane (also see above).

α, β, γ: These terms are used in their conventional sense, to refer to the relative position of bonds, atoms or substituents within a molecule. The position described as α to a particular atom or group is one bond away from it, β is two bonds away, and so on, as illustrated below using a carbonyl compound.

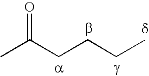

Includes Other Forms

Unless otherwise specified, included in the above are the well known ionic, solvate, and protected forms of these substituents. For example, a reference to carboxylic acid (—COOH) also includes the anionic (carboxylate) form (—COO⁻) or solvate thereof, as well as conventional protected forms. Similarly, a reference to an amino group includes the protonated form (—N⁺HR¹R²) or solvate of the amino group, as well as conventional protected forms of an amino group. Similarly, a reference to a hydroxyl group also includes the anionic form (—O⁻) or solvate thereof, as well as conventional protected forms.

Isomers

Certain compounds may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: ketolenol (illustrated below), imine/enamine, amide/imino alcohol, amidine/amidine, nitroso/oxime, thioketone/enethiol, N-nitroso/hydroxyazo, and nitro/aci-nitro.

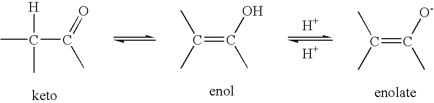

In particular it will be understood that the azo-containing ligands of the present invention may exist in more than one tautomeric form and that the predominant form may change upon coordination to the metal. For example a ligand such as the example below can be drawn in two different forms:

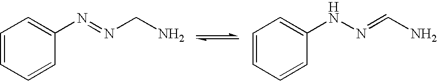

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof, for example, a mixture enriched in one enantiomer. Methods for the preparation (e.g., asymmetric synthesis) and separation (e.g., fractional crystallization and chromatographic means) of such isomeric forms are either known in the art or are readily obtained by adapting the methods taught herein, or known methods, in a known manner.

Solvates

It may be convenient or desirable to prepare, purify, and/or handle a corresponding solvate of the active compound. The term “solvate” is used herein in the conventional sense to refer to a complex of solute (e.g., active compound, salt of active compound) and solvent. If the solvent is water, the solvate may be conveniently referred to as a hydrate, for example, a mono-hydrate, a di-hydrate, a tri-hydrate, etc.

Unless otherwise specified, a reference to a particular compound also include solvate forms thereof.

Chemically Protected Forms

It may be convenient or desirable to prepare, purify, and/or handle the active compound in a chemically protected form. The term “chemically protected form” is used herein in the conventional chemical sense and pertains to a compound in which one or more reactive functional groups are protected from undesirable chemical reactions under specified conditions (e.g., pH, temperature, radiation, solvent, and the like). In practice, well known chemical methods are employed to reversibly render unreactive a functional group, which otherwise would be reactive, under specified conditions. In a chemically protected form, one or more reactive functional groups are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts; 3rd Edition; John Wiley and Sons, 1999).

Unless otherwise specified, a reference to a particular compound also includes chemically protected forms thereof.

A wide variety of such “protecting,” “blocking,” or “masking” methods are widely used and well known in organic synthesis. For example, a compound which has two nonequivalent reactive functional groups, both of which would be reactive under specified conditions, may be derivatized to render one of the functional groups “protected,” and therefore unreactive, under the specified conditions; so protected, the compound may be used as a reactant which has effectively only one reactive functional group. After the desired reaction (involving the other functional group) is complete, the protected group may be “deprotected” to return it to its original functionality.

For example, a hydroxy group may be protected as an ether (—OR) or an ester (—OC(═O)R), for example, as: a t-butyl ether; a benzyl, benzhydryl (diphenylmethyl), or trityl (triphenylmethyl) ether; a trimethylsilyl or t-butyldimethylsilyl ether; or an acetyl ester (—OC(═O)CH₃, —OAc).

For example, an aldehyde or ketone group may be protected as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide (—NRCO—R) or a urethane (—NRCO—OR), for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulphonyl)ethyloxy amide (—NH-Psec); or, in suitable cases (e.g., cyclic amines), as a nitroxide radical (>N—O).

For example, a carboxylic acid group may be protected as an ester for example, as: an C₁₋₇alkyl ester (e.g., a methyl ester; a t-butyl ester); a C₁₋₇haloalkyl ester (e.g., a C₁₋₇trihaloalkyl ester); a triC₁₋₇alkylsilyl-C₁₋₇alkyl ester; or a C₅₋₂₀aryl-C₁₋₇alkyl ester (e.g., a benzyl ester; a nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃).

Prodrugs

It may be convenient or desirable to prepare, purify, and/or handle the active compound in the form of a prodrug. The term “prodrug,” as used herein, pertains to a compound which, when metabolized (e.g., in vivo), yields the desired active compound. Typically, the prodrug is inactive, or less active than the active compound, but may provide advantageous handling, administration, or metabolic properties.

Unless otherwise specified, a reference to a particular compound also include prodrugs thereof.

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include those of the formula —C(═O)OR wherein R is:

C₁₋₇alkyl

(e.g., -Me, -Et, -nPr, -iPr, -nBu, -sBu, -iBu, -tBu);

C₁₋₇aminoalkyl

(e.g., aminoethyl; 2-(N,N-diethylamino)ethyl; 2-(4-morpholino)ethyl); and

acyloxy-C₁₋₇alkyl

(e.g., acyloxymethyl;

acyloxyethyl;

pivaloyloxymethyl;

acetoxymethyl;

1-acetoxyethyl;

1-(1-methoxy-1-methyl)ethyl-carbonxyloxyethyl;

1-(benzoyloxy)ethyl; isopropoxy-carbonyloxymethyl;

1-isopropoxy-carbonyloxyethyl; cyclohexyl-carbonyloxymethyl;

1-cyclohexyl-carbonyloxyethyl;

cyclohexyloxy-carbonyloxymethyl;

1-cyclohexyloxy-carbonyloxyethyl;

(4-tetrahydropyranyloxy)carbonyloxymethyl;

1-(4-tetrahydropyranyloxy)carbonyloxyethyl;

(4-tetrahydropyranyl)carbonyloxymethyl; and

1-(4-tetrahydropyranyl)carbonyloxyethyl).

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

Use of Compounds of the Invention

The invention provides compounds of formula (I), or solvates or prodrugs thereof (“active compounds”), for use in a method of treatment of the human or animal body. Such a method may comprise administering to such a subject a therapeutically-effective amount of an active compound, preferably in the form of a pharmaceutical composition.

The term “treatment” as used herein in the context of treating a condition, pertains generally to treatment and therapy, whether of a human or an animal (e.g. in veterinary applications), in which some desired therapeutic effect is achieved, for example, the inhibition of the progress of the condition, and includes a reduction in the rate of progress, a halt in the rate of progress, amelioration of the condition, and cure of the condition. Treatment as a prophylactic measure (i.e. prophylaxis) is also included.

The term “therapeutically-effective amount” as used herein, pertains to that amount of an active compound, or a material, composition or dosage form comprising an active compound, which is effective for producing some desired therapeutic effect, commensurate with a reasonable benefit/risk ratio.

Administration

The active compound or pharmaceutical composition comprising the active compound may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly.

The subject may be a eukaryote, an animal, a vertebrate animal, a mammal, a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orang-utan, gibbon), or a human.

Formulations

While it is possible for the active compound to be administered alone, it is preferable to present it as a pharmaceutical composition (e.g. formulation) comprising at least one active compound, as defined above, together with one or more pharmaceutically acceptable carriers, adjuvants, excipients, diluents, fillers, buffers, stabilizers, preservatives, lubricants, or other materials well known to those skilled in the art and optionally other therapeutic or prophylactic agents.

Thus, the present invention further provides pharmaceutical compositions, as defined above, and methods of making a pharmaceutical composition comprising admixing at least one active compound, as defined above, together with one or more pharmaceutically acceptable carriers, excipients, buffers, adjuvants, stabilizers, or other materials, as described herein.

The term “pharmaceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, excipients, etc. can be found in standard pharmaceutical texts, for example, Remington's Pharmaceutical Sciences, 18th edition, Mack Publishing Company, Easton, Pa., 1990.

The formulations may conveniently be presented in unit dosage form and may be prepared by any methods well known in the art of pharmacy. Such methods include the step of bringing into association the active compound with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, losenges, granules, powders, capsules, cachets, pills, ampoules, suppositories, pessaries, ointments, gels, pastes, creams, sprays, mists, foams, lotions, oils, boluses, electuaries, or aerosols.

Formulations suitable for oral administration (e.g. by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g., compression or moulding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g. sodium lauryl sulfate); and preservatives (e.g. methyl p-hydroxybenzoate, propyl p-hydroxybenzoate, sorbic acid). Moulded tablets may be made by moulding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for topical administration (e.g. transdermal, intranasal, ocular, buccal, and sublingual) may be formulated as an ointment, cream, suspension, lotion, powder, solution, past, gel, spray, aerosol, or oil. Alternatively, a formulation may comprise a patch or a dressing such as a bandage or adhesive plaster impregnated with active compounds and optionally one or more excipients or diluents.

Formulations suitable for topical administration in the mouth include losenges comprising the active compound in a flavoured basis, usually sucrose and acacia or tragacanth; pastilles comprising the active compound in an inert basis such as gelatin and glycerin, or sucrose and acacia; and mouthwashes comprising the active compound in a suitable liquid carrier.

Formulations suitable for topical administration to the eye also include eye drops wherein the active compound is dissolved or suspended in a suitable carrier, especially an aqueous solvent for the active compound.

Formulations suitable for nasal administration, wherein the carrier is a solid, include a coarse powder having a particle size, for example, in the range of about 20 to about 500 microns which is administered in the manner in which snuff is taken, i.e. by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the active compound.

Formulations suitable for administration by inhalation include those presented as an aerosol spray from a pressurized pack, with the use of a suitable propellant, such as dichlorodifluoromethane, trichlorofluoromethane, dichoro-tetrafluoroethane, carbon dioxide, or other suitable gases.

Formulations suitable for topical administration via the skin include ointments, creams, and emulsions. When formulated in an ointment, the active compound may optionally be employed with either a paraffinic or a water-miscible ointment base. Alternatively, the active compounds may be formulated in a cream with an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

When formulated as a topical emulsion, the oily phase may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Suitable emulgents and emulsion stabilizers include Tween 60, Span 80, cetostearyl alcohol, myristyl alcohol, glyceryl monostearate and sodium lauryl sulphate. The choice of suitable oils or fats for the formulation is based on achieving the desired cosmetic properties, since the solubility of the active compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required.

Alternatively, high melting point lipids such as white soft paraffin and/or liquid paraffin or other mineral oils can be used.

Formulations suitable for rectal administration may be presented as a suppository with a suitable base comprising, for example, cocoa buffer or a salicylate.

Formulations suitable for vaginal administration may be presented as pessaries, tampons, creams, gels, pastes, foams or spray formulations containing in addition to the active compound, such carriers as are known in the art to be appropriate.

Formulations suitable for parenteral administration (e.g. by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilizers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

Dosage

It will be appreciated that appropriate dosages of the active compounds, and compositions comprising the active compounds, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g. in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In general, a suitable dose of the active compound is in the range of about 100 μg to about 250 mg per kilogram body weight of the subject per day. Where the active compound is a salt, an ester, prodrug, or the like, the amount administered is calculated on the basis of the parent compound and so the actual weight to be used is increased proportionately.

Cancers

Examples of cancers which may be treated by the active compounds include, but are not limited to, a carcinoma, for example a carcinoma of the bladder, breast, colon (e.g. colorectal carcinomas such as colon adenocarcinoma and colon adenoma), kidney, epidermal, liver, lung, for example adenocarcinoma, small cell lung cancer and non-small cell lung carcinomas, oesophagus, gall bladder, ovary, pancreas e.g. exocrine pancreatic carcinoma, stomach, cervix, thyroid, prostate, or skin, for example squamous cell carcinoma; a hematopoietic tumour of lymphoid lineage, for example leukemia, acute lymphocytic leukemia, B-cell lymphoma, T-cell lymphoma, Hodgkin's lymphoma, non-Hodgkin's lymphoma, hairy cell lymphoma, or Burkett's lymphoma; a hematopoietic tumor of myeloid lineage, for example acute and chronic myelogenous leukemias, myelodysplastic syndrome, or promyelocytic leukemia; thyroid follicular cancer; a tumour of mesenchymal origin, for example fibrosarcoma or habdomyosarcoma; a tumor of the central or peripheral nervous system, for example astrocytoma, neuroblastoma, glioma or schwannoma; melanoma; seminoma; teratocarcinoma; osteosarcoma; xenoderoma pigmentoum; keratoctanthoma; thyroid follicular cancer; or Kaposi's sarcoma.

Examples of other therapeutic agents that may be administered together (whether concurrently or at different time intervals) with the compounds of the formula (I) include but are not limited to topoisomerase inhibitors, alkylating agents, antimetabolites, DNA binders and microtubule inhibitors (tubulin target agents), such as cisplatin, cyclophosphamide, doxorubicin, irinotecan, fludarabine, 5FU, taxanes, mitomycin C or radiotherapy. For the case of active compounds combined with other therapies the two or more treatments may be given in individually varying dose schedules and via different routes.

The combination of the agents listed above with a compound of the present invention would be at the discretion of the physician who would select dosages using his common general knowledge and dosing regimens known to a skilled practitioner.

Where the compound of the formula (I) is administered in combination therapy with one, two, three, four or more, preferably one or two, preferably one other therapeutic agents, the compounds can be administered simultaneously or sequentially. When administered sequentially, they can be administered at closely spaced intervals (for example over a period of 5-10 minutes) or at longer intervals (for example 1, 2, 3, 4 or more hours apart, or even longer periods apart where required), the precise dosage regimen being commensurate with the properties of the therapeutic agent(s).

The compounds of the invention may also be administered in conjunction with non-chemotherapeutic treatments such as radiotherapy, photodynamic therapy, gene therapy; surgery and controlled diets.

Preferences

Preferably the compounds of formula (I) are monomeric. If the compounds of formula (I) are in the form of a dimer the linking group is preferably phenylene (e.g. phenyl-4-ene), C₁₋₃ alkylene, —NH— or —O— and more preferably phenylene (e.g. phenyl-4-ene), C₁₋₃ alkylene or —O—. If the linking group links two B groups, these may preferably be C₅₋₂₀ aryl (e.g. phenyl). If one group serves a B for both moieties, this is preferably phenylene (e.g. 4-phenylene).

R¹-R⁶

In one group of embodiments of the present invention, R¹ and R² together with the ring to which they are attached form a saturated or unsaturated carbocyclic or heterocyclic group containing up to 3- to 8-membered carbocyclic or heterocyclic rings, wherein each carbocyclic or heterocyclic ring may be fused to one or more other carbocyclic or heterocyclic rings.

In this group of embodiments, it is preferred that R³, R⁴, R⁵ and R⁶ are H.

R¹ and R² together with the ring to which they are bound in compounds of formula (I) may represent an ortho- or peri-fused carbocyclic or heterocyclic ring system.

R¹ and R² together with the ring to which they are bound may represent a wholly carbocyclic fused ring system such as a ring system containing 2 or 3 fused carbocyclic rings, e.g. optionally substituted, optionally hydrogenated naphthalene or anthracene.

Alternatively, R¹ and R² together with the ring to which they are bound in compounds of formula (I) may represent a fused tricyclic ring such as anthracene or a mono, di, tri, tetra or higher hydrogenated derivative of anthracene. For example, R¹ and R² together with the ring to which they are bound in formula (I) may represent anthracene, 1,4-dihydroanthracene or 1,4,9,10-tetrahydroanthracene.

R¹ and R² together with the ring to which they are bound in formula (I) may also represent:

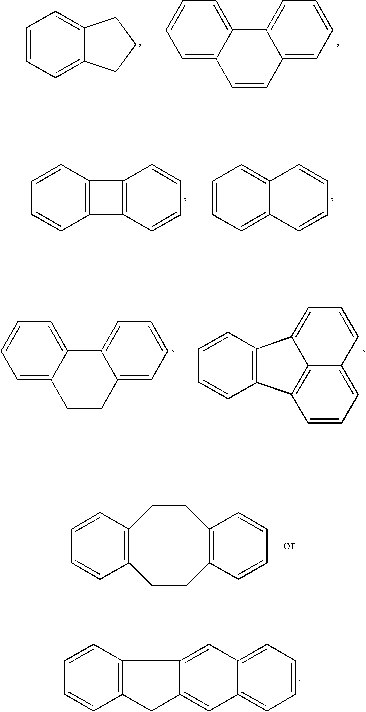

In another group of embodiments, R¹, R², R³, R⁴, R⁵ and R⁶ are independently selected from H, C₁₋₇ alkyl, C₅₋₂₀ aryl, C₃₋₂₀ heterocyclyl, halo, ester, amido, acyl, sulfo, sulfonamido, ether, thioether, azo and amino. In this group of embodiments, R¹, R², R³, R⁴, R⁵ and R⁶ are preferably independently selected from H, C₁₋₇ alkyl, C₅₋₂₀ aryl and ester. Of these H and C₁₋₇ alkyl (in particular C₁₋₃ alkyl)are most preferred.

In this group of embodiments, four, five or six of R¹, R², R³, R⁴, R⁵ and R⁶ are preferably hydrogen, with the other (if any) groups being selected from C₁₋₇ alkyl, C₅₋₂₀ aryl, C₃₋₂₀ heterocyclyl, halo, ester, amido, acyl, sulfo, sulfonamido, ether, thioether, azo and amino, or more preferably C₁₋₇ alkyl, C₅₋₂₀ aryl and ester, and most preferably C₁₋₇ alkyl (in particular C₁₋₃ alkyl). If two of R¹, R², R³, R⁴, R⁵ and R⁶ are not H, then these groups are preferably meta or para to one another, and more preferably para to one another.

Examples of particularly preferred substituent patterns include, but are not limited to: phenyl; 1-methyl; and 4-iso-propyl.

A

It is preferred that A is a nitrogen containing aromatic ring, wherein the nitrogen ring atom is bound to the ruthenium atom, and the ring is further bound to the azo-nitrogen, either by a single bond α or β to the nitrogen ring atom, or by a —CH₂-group α to the nitrogen ring atom.

The nitrogen containing aromatic ring is preferably unsubstituted.

In the case that A is —NR^{N1}R^{N2}—R^{N3}—, it is preferred that R^{N1} and R^{N2} are independently selected from H, C₁₋₇ alkyl or C₅₋₂₀ aryl. It is more preferable that at least one of R^{N1} and R^{N2} is H. Most preferably R^{N1} and R^{N2} are both H.

More preferably, the ring is bound to the azo-nitrogen by a single bond a to the nitrogen ring atom. It is further preferred that the nitrogen-containing aromatic ring is pyridine or pyrazole.

B

It is preferred that B is optionally substituted C₅₋₂₀ aryl or optionally substituted C₁₋₇ alkyl. More preferably B represents substituted or unsubstituted phenyl, or benzyl. If B is substituted phenyl, it is most preferably substituted with a group selected from —OR^{O1}, —NR^{N1}R^{N2}, —NO₂, C₁₋₇ alkyl, C₅₋₂₀ aryl, wherein R^{O1}, R^{N1} and R^{N2} are independently selected from H, C₁₋₇ alkyl, C₃₋₂₀ heterocyclyl or C₅₋₂₀ aryl. More preferably, B is phenyl substituted with —OR^{O1} or —NR^{N1}R^{N2}, wherein R^{O1}, R^{N1} and R^{N2} are independently H or C₁₋₇ alkyl. It is further preferred that the substitution is in the para position. R^{O1} is more preferably H. R^{N1} and R^{N2} are more preferably methyl.

X

X is preferably halo and is more preferably I or Cl.

Y^{q-}

Y^{q-} in compounds of formula (I) is a counterion and is only present in the compound when the complex containing the metal ion is charged. Y^{q-} is preferably a non-nucleophilic anion such as PF₆⁻, BF₄⁻, BPh₄⁻ or CF₃O₂SO⁻, for example. It may also be I⁻.

General Synthesis Methods

The present invention also provides a process for preparing the compounds of the invention which comprises the reaction of a dimeric ruthenium complex of formula [(η⁶-C₆(R¹)(R²)(R³)(R⁴)(R⁵)(R⁶))RuX₂]₂ with a ligand of formula AN═NB in the presence, or with subsequent addition of, Y^{q-}, in a suitable solvent for the reaction, wherein R¹, R², R³, R⁴, R⁵, R⁶, X, A, B and Y are as defined above for the compounds of the invention.

Preferred reaction conditions include:
(a) stirring the starting dimeric ruthenium complex, as described above, in MeOH or a MeOH/water mixture;
(b) adding the ligand as a solution in MeOH;
(c) stirring the resultant solution at room temperature; and
(d) adding a source of Y^{q-}, such as a compound of formula (NH₄⁺)Y^{q-}, e.g., NH₄PF₆, and filtering off the precipitated product.

Dimeric compounds may be made in an analogous manner, using techniques described in the art.

### FIGURES

FIG. 1 shows a 1H 2D TOCSY of a compound of the invention during a hydrolysis experiment.

FIG. 2 shows the absorbance over time of a reaction between a compound of the invention and ascorbate.

FIG. 3 shows the NMR spectra over time of the same reaction as in FIG. 2.

FIG. 4 shows the change in DCF fluorescence over time upon exposure to compounds of the invention and a comparative compound.

FIG. 5 shows the percentage cell survival of A549 cancer cells.

The following non-limiting examples illustrate the present invention.

### EXAMPLES

General Methods

Materials: The starting materials [(η⁶-arene)RuCl₂]₂ (arene=p-cymene, tetrahydronapthalene (THN), benzene, biphenyl, hydroxyethoxybenzene) were prepared according to the literature (Bennett, M. A., Smith, A. K., J. Chem. Soc. Dalton Trans. 1974, (2), 233-241; Zelonka, R. A., Baird, M. C., J. Organometallic Chem., 1972, 35, (1), C43-C46; Soleimannejad, J. & White, C., 2005, Organometallics, 24, 2538-2541). N,N-Dimethyl-4-(2-pyridylazo)aniline (Azpy-NMe₂), aniline, NaNO₂, 2-cyanoethylhydrazine, N,N-dimethylaniline, ortho-phosphoric acid, benzoquinone, 2-hydrazinopyridine and NOHSO₄ were purchased from Sigma-Aldrich and were used as received. The ethanol used was dried over Mg/I₂ and the methanol used was either dried over Mg/I₂ or anhydrous quality was used (Sigma-Aldrich). The ruthenium standard (1000 ppm) was purchased from Sigma Aldrich. All other reagents used were obtained from commercial suppliers and used as received

NMR-Spectroscopy: All ¹H NMR experiments for characterization of synthesized compounds were recorded on either a Bruker DMX 500 MHz spectrometer equipped with TBI [¹H, ¹³C, ¹⁵N] probe-head, equipped with z-field gradients or a Bruker DPX 360 MHz spectrometer. The proton signals were calibrated against the residual solvent peak, δ 7.27 (chloroform), 2.07 (acetone) and 2.52 (DMSO). The 2D ¹H-TOCSY and 2D ¹H COSY experiments for characterization were run on a Bruker DMX 500 MHz spectrometer. 2D-¹H ROESY experiment for characterization was recorded on a Bruker AVA 600 mHz spectrometer equipped with a with a TXI [¹H, ¹³C, ¹⁵N] probe-head, equipped with z-field gradients. All pH titration experiments were recorded on a Bruker AVA 600 MHz spectrometer where dioxan was added as an internal reference (δ 3.75, in 100% D₂O). The water was suppressed using a 1D Double Pulse Field Gradient Spin Echo (DPFGSE) experiment. The aqueous solution behaviour was recorded on a Bruker bio 600 MHz spectrometer equipped with a cryoprobe and the water was suppressed using a 1D Double Pulse Field Gradient Spin Echo (DPFGSE) experiment. The chemical shifts were measured relative to dioxin (internal reference δ 3.75, in 90% H₂O/10% D₂O). All spectra were recorded using 5 mm quartz tubes at 298 K unless stated otherwise. All NMR data were processed using Xwin-NMR (Version 2.0 Bruker UK Ltd).

Elemental Analysis: Elemental analysis was carried out by the University of Edinburgh using an Exeter analytical elemental analyzer CE440.

Electrospray Mass Spectrometry: ESI-MS were obtained on a Micromass Platform II Mass Spectrometer and solutions were infused directly. The capillary voltage was 3.5 V and the cone voltage used was dependent on the solution (typically varied between 5-15 V). The source temperature was ca. 383 K.

ICP-AES: Ruthenium content in aqueous solutions was determined by ICP-AES using a Thermo Jarrell Ash IRIS ICP-AES machine. Ruthenium standards were first run to give a calibration curve and the ruthenium concentration was measured by emission at 240.272 and 349.549 nm relative to this calibration.

Synthesis of Iodide Dimers

These are readily synthesized from the corresponding chloride dimers by addition of excess KI in water.

### [(η⁶-p-cymene)RuI₂]₂

The dimer [(η⁶-p-cymene))RuCl₂]₂ (0.65 g, 1 mmol) was refluxed in water (250 ml) for 1 hour. The solution was hot filtered and KI (4.45 g, 27 mmol) was added to the filtrate. A brown/red precipitate immediately formed. This was filtered off and washed with ethanol and ether. Yield: 841 mg (86.0%) ¹H NMR (DMSO-d6) δ 5.87 (d of d, 4H), 3.16 (septet, 1H), 2.40 (s, 3H), 1.22 (d, 6H).

### [(η⁶-biphenyl)RuI₂]₂

The dimer [(η⁶-biphenyl)RuCl₂]₂ (0.3 g, 0.46 mmol ) was stirred at ambient temperature in water (250 ml) for 30 minutes. The solution was filtered and KI (2.12 g, 13 mmol) was added to the filtrate. A brown/red precipitate immediately formed. This was filtered off and washed with ethanol and ether. Yield: 388 mg (82.9%) ¹H NMR (DMSO-d6) δ 7.84 (d, 2H), 7.54-7.46 (m, 3H), 6.66 (d, 2H), 6.38 (t, 1H), 6.12 (d, 2H).

Synthesis of Ligands

The chelating azo ligands used were synthesized according to previously published procedures (Suminov, S. I., Zhurnal Organicheskoi Khimii, 1968, 4, (10), 1864-5; Gorelik, M. V.; Lomzakova, V. I., Zhumal Organicheskoi Khimii, 1986, 22, (5), 1054-61; Betteridge, D.; John, D., Analyst (Cambridge, United Kingdom), 1973, 98 (1167), 377-89; Krause, R. A.; Krause, K., Inorganic Chemistry, 1980, 19, (9), 2600-3) and were characterized by NMR and ESI-MS.

### 2-Phenylazopyridine (1)

A portion of 2-aminopyridine (5.49 g, 0.0583 mol) was added to NaOH (27.06 g, 0.677 mol) in 30 ml water containing 5 ml benzene. Over a 15-minute period nitrosobenzene (6.08 g, 0.0567 mol) was added whilst the mixture was warmed on an oil bath. The mixture was heated under reflux for a further 10 minutes and was then extracted three times with 100 ml portions of toluene. The organic layer collected was dried with magnesium sulfate and treated with decolourizing charcoal. The toluene was removed on a rotary evaporator and the solid obtained was dried in vacuo under argon. The solid was dissolved in 500 ml of hot petroleum ether (40-60° C.) and a brown residue was decanted. The solution was cooled in a container of dry ice overnight. The recrystallization step was repeated twice and in these cases the volume of petroleum ether used for recrystallization was reduced to 50 ml. A red solid was obtained and used for further synthesis without further purification. Yield: 3.773 g (36.3%). ¹H NMR (CDCl₃): δ 8.76 (1H, d), 8.08-8.05 (2H, m), 7.93 (1H, t, 7.85 (1H, d) (3H, m), 7.42 (1H, t,) ESI-MS: m/z 184.2 (M+).

### 4-Phenol-azo pyridine (2)

Benzoquinone (0.493 g, 4.56 mmol) was dissolved in a solution of 50 ml water and 3.6 ml 60% perchloric acid. Hydrazinopyridine (0.504 g, 4.62 mmol) dissolved in 8 ml water was added dropwise and the solution gradually turned brown/orange. The solution was stirred at room temperature for one hour and filtered to leave an orange crystalline precipitate. The precipitate was dissolved in 25 ml methanol and 1.5 ml formic acid and ammonia gas was bubbled through the mixture until reprecipitation occurred. The product was filtered and left to dry overnight in vacuo. A second crop was obtained by reducing the volume of the solvent of the filtrate. Yield 213 mg (23.36%). ¹H NMR (DMSO-d₆) δ 8.73 (d, 1H), 8.13 (t, 1H), 7.91 (d, 2H), 7.75 (d, 1H), 7.61 (t, 1H), 7.01 (d, 2H). ESI-MS: m/z 200.2 (M⁺)

### 3-Amino-pyrazoline hydrochloride (3)

Sodium metal (0.096 g, 4.17 mmol) was added to 15 ml dry ethanol. 2-Cyanoethylhydrazine (2.5 g, 29.4 mmol) was added dropwise and the mixture was heated under reflux for four hours. The reaction mixture was left to cool and 50 ml 37% HCl was added dropwise. The reaction mixture turned green/yellow and a white precipitate formed at the bottom of the flask. The flask was kept cool by surrounding in ice and the solution was filtered through a frit under suction. The crude product was dissolved in acidified water where NaCl impurities precipitated out. These salts were removed by filtration, water was removed on the rotary evaporator and the white solid product was dried overnight in vacuo. Yield: 2.48 g (68%). ¹H NMR (DMSO-d₆) δ 7.09 (br s, 1H), 3.42 (t, 2H), 2.85 (t, 2H)

### 3-Amino-1-nitroso-2-pyrazoline (4)

3-Amino-pyrazoline hydrochloride (3) (1 g, 8.23 mmol) was suspended in 8 ml acetic acid and the flask was cooled by surrounding in ice. NaNO₂ (0.57 g, 8.23 mmol) was dissolved in 1 ml water and added dropwise to the cooled solution over 70 minutes. The solution was stirred at 0° C. for four hours. The solvent was removed and the orange solid was re-dissolved in 3 ml water. The flask was kept cold by surrounding in ice and the mixture was filtered under suction. The orange powder obtained was dried overnight in vacuo. Yield: 204 mg (22%). ESI-MS: m/z 114.6 (M+), 84.6 (M-NO).

### 3(5)-(4-Dimethylaminophenylazo)pyrazole (5)

3-Amino-1-nitroso-2-pyrazoline (4) (353 mg, 3.07 mmol) was dissolved in 3 ml o-phosphoric acid and stirred at 25° C. 2.4 ml of 18M H₂SO₄ was added slowly to this mixture so that the temperature did not exceed ca. 313 K. Once the mixture has stopped bubbling a solution of 0.98 g of 40% wt NOHSO₄ in 0.98 g H₂SO₄ was added over one hour. The reaction was subsequently stirred at 48-50° C. for one and a half hours then poured onto 35 g ice. N,N-dimethylaniline (0.361 g, 3 mmol) was dissolved in 20 ml water. To this solution the diazotized mixture was added dropwise and the pH was kept between 4 and 5 by addition of sodium carbonate (saturated solution). The yellow solution was filtered and the precipitate was washed with water. The yellow solid was dried overnight in vacuo. Yield: 375 mg (57%). ¹H NMR (CDCl₃) ¹H NMR (D₂O) δ8.05 (d, 2H), 7.84 (d, 1H), 7.75 (d, 2H), 6.76 (d, 1h), 3.37 (s, 6H). ESI-MS: m/z 215.5 (M⁺).

### N,N-Dimethyl-4-(2-pyridylazo)-1-nitro-aniline (6)

N,N-Dimethyl-4-(2-pyridylazo)aniline (Azpy-NMe₂, 200 mg, 0.88 mmol) was placed in a flask and cooled in ice/salt/water. 0.42 ml of 18M H₂SO₄ was added dropwise with stirring and the mixture was subsequently stirred for one hour. A solution of 0.56 ml of 70% HNO₃ and 0.56 ml of 18M H₂SO₄ was cooled in ice/salt/water and added dropwise to the mixture and left to stir for two hours, with constant cooling. 0.06 ml ice water was added followed by dropwise addition of 0.45 ml of 45% NH₃OH to quench. The product was extracted from the aqueous layer into chloroform. The solvent was removed to leave an oily product, which was dissolved in the minimum volume of ether and scratched to give an orange solid. The crude product was purified by column chromatography using 50:50 ethyl acetate: hexane as the eluting solvent and silica. Yield: 60.5 mg (25.34%). ¹H HMR (CDCl₃) δ 8.74 (s, 1H), 8.57 (d, 1H), 8.18 (d of d, 1H), 7.92 (t, 1H), 7.83 (d, 1H), 7.41 (t, 1H), 7.11 (d, 1H). ESI-MS: m/z 271.6 (M+)

Synthesis of Ruthenium Complexes

Some complexes of the present invention can be represented by structures (i) and (ii), below.

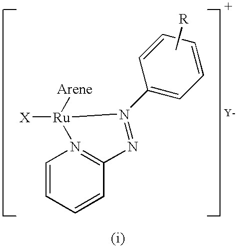

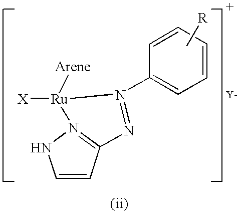

| Complex | Type | Arene | X | R | Y |
|---|---|---|---|---|---|
| 7 (SD028) | (i) | biphenyl | I | H | PF₆ |
| 8 (SDPR9) | (i) | p-cymene | I | p-NMe₂ | PF₆ |
| 9 (SDPR7) | (i) | biphenyl | Cl | p-NMe₂ | PF₆ |
| 10 (SD024) | (i) | biphenyl | I | p-NMe₂ | PF₆ |
| 11 (SD010) | (i) | p-cymene | Cl | p-OH | PF₆ |
| 12 (SD016) | (i) | biphenyl | Cl | p-OH | PF₆ |
| 13 (SD026) | (i) | biphenyl | I | p-OH | PF₆ |
| 14 (SD002) | (ii) | p-cymene | Cl | p-NMe₂ | PF₆ |
| 15 (SD006) | (ii) | biphenyl | Cl | p-NMe₂ | PF₆ |
| 16 (SD025) | (ii) | biphenyl | I | p-NMe₂ | PF₆ |
| 17 (SD005) | (ii) | benzene | Cl | p-NMe₂ | PF₆ |
| 18 (SD014) | (ii) | tetrahydronaphthalene | Cl | p-NMe₂ | PF₆ |
| 19 (SD012) | (i) | tetrahydronaphthalene | Cl | p-OH | PF₆ |
| 20 (SD018) | (i) | benzene | Cl | m-NO₂, | PF₆ |
| | | | | p-NMe₂ | |
| 21 (SD029) | (i) | p-cymene | I | p-OH | PF₆ |
| 22 (SD040) | (i) | hydroxyethoxybenzene | I | p-NMe₂ | I |

### [Ru(bip)(2-phenyl-azo pyridine)I]PF₆ (7)

The dimer [RuI₂(biphenyl)]₂ (100 mg, 0.1 mmol) was dissolved in 80 ml 75% methanol and heated to reflux for two hours. 2-phenylazopyridine (1) (37.5 mg, 0.2 mmol) dissolved in 20 ml methanol was added drop-wise and the solution gradually turned from brown to brown/purple. The solution refluxed for a further two hours, hot filtered and then the volume of solvent was reduced to about 15 ml by removal of methanol on a rotary evaporator. NH₄PF₆ (160 mg, 1 mmol) was then added and the solution was placed in the fridge for two hours. A black powder product precipitated out and this was filtered off and washed with cold ethanol then ether. Yield 94.1 mg (66.1%). ¹H NMR (DMSO-d₆) δ 9.45 (d, 1H), 8.93 (d, 1H), 8.41 (t, 1H), 7.94 (d, 2H), 7.64-7.56 (m, 3H), 7.60-7.45 (m, 5H), 7.43-7.33 (m, 2H), 6.89 (d, 1H), 6.75 (t, 1H), 6.70-6.54 (m, 3H). ESI-MS m/z 566.1 (M⁺).

### [Ru(p-cymene)(Azpy-NMe₂)I]PF₆ (8)

The dimer [RuI₂(p-cymene)]₂ (54.8 mg, 0.051 mmol) was dissolved in 20 ml methanol and heated to approximately 40° C. until the solution turned clear. Azpy-NMe₂ (23 mg, 0.102 mmol) dissolved in 10 ml methanol was added drop-wise and the solution immediately turned from brown to dark blue. The solution was stirred at room temperature for three hours. The volume of solvent was reduced to about 10 ml by removal of methanol on a rotary evaporator. NH₄PF₆ (83 mg, 0.51 mmol) was then added and the solution was placed in the freezer overnight. A black microcrystalline product precipitated out and this was filtered off and washed with ether. Yield: 40.9 mg (68.2%) (Found: C, 37.74; H, 3.25; N, 7.40. Calc for RuC₂₃H₂₇N₄IPF₆: C, 37.66; H, 3.85; N, 7.64). ¹H NMR (CDCl₃) δ 9.17 (d, 1H), 8.22 (d, 1H), 8.15 (d, 2H), 8.03 (t, 1H), 7.54 (t, 1H), 6.77 (d, 2H), 6.05 (d, 1H), 5.81 (t, 2H), 5.68 (d, 1H), 3.29 (s, 6H), 2.70-2.54 (m, 4H), 1.04 (d of d, 6H). ESI-MS m/z 589.3 (M⁺).

### [Ru(biphenyl)(Azpy-NMe₂)Cl]PF₆ (9)

The dimer [RuCl₂(biphenyl)]₂ (105.1 mg, 0.161 mmol) was dissolved in a solution of 40 ml methanol and 10 ml water. The solution was refluxed under argon for 2 hours. Azpy-NMe₂ (78.15 mg, 0.345 mmol) dissolved in 5 ml methanol was added drop-wise and the solution immediately turned from brown to very dark blue. The mixture was hot filtered and left to cool to room temperature whilst stirring. After thirty minutes, the volume of solvent was reduced to about 15 ml by removal of methanol on a rotary evaporator. NH₄PF₆ (187 mg, 1.14 mmol) was then added and the solution was left in the fridge overnight. The black crystalline powder precipitated out and was filtered off and washed with methanol until the filtrate turned blue. Yield: 130 mg (61.1%) (Found: C, 45.31; H, 3.56; N, 8.44. Calc for RuC₂₅H₂₄N₄ClPF₆: C, 45.31; H, 3.61; N, 8.46). ¹H NMR ((CD₃)₂CO): δ 9.25 (1H, d), 8.36 (1H, d), 8.29 (1H, t), 8.22 (2H, d), 7.75-7.71 (2H, m), 7.60-755 (2H, m), 7.54-7.48 (2H, t), 6.91 (2H, d), [6.75 (1H, d), 6.65 (1H, d), 6.57 (2H, d of d), 6.38 (1H, t), 3.36 (6H, s).

### [Ru(biphenyl)(Azpy-NMe₂)Cl]PF₆ (10)

The dimer [RuI₂(biphenyl)]₂ (100 mg, 0.1 mmol) was dissolved in 80 ml 75% methanol and heated to reflux for two hours. Azpy-NMe₂ (44.4 mg, 0.2 mmol) dissolved in 20 ml methanol was added drop-wise and the solution immediately turned from brown to dark blue. The solution was refluxed for a further hour, hot filtered and then the volume of solvent was reduced to about 15 ml by removal of methanol on a rotary evaporator. NH₄PF₆ (160 mg, 1 mmol) was then added and the solution was placed in the fridge for one hour. A black powder product precipitated out and this was filtered off and washed with cold ethanol then ether. Yield 121 mg (80.2%). ¹H NMR (DMSO-d₆) δ9.35 (d, 1H), 8.37 (d, 1H), 8.15 (t, 1H), 8.07 (d, 2H), 7.51-7.34 (m, 5H), 6.86-6.78 (m, 3H), 6.68-6.48 (m, 4H), 3.27 (s, 6H). ESI-MS m/z 602.9 (M⁺).

### [Ru(p-cymene)(4-phenol-azo pyridine)Cl]PF₆ (11)

The dimer [RuCl₂(p-cymene)]₂ (40 mg, 0.048 mmol) was dissolved in 15 ml methanol and left to stir at room temperature until the solution turned clear. 4-Phenol-azo pyridine (2) (21 mg, 0.096 mmol) dissolved in 10 ml methanol was added drop-wise and the solution gradually turned from brown to deep brown/red with a yellow tinge. The solution was stirred at room temperature for three hours. The volume of solvent was reduced to about 10 ml by removal of methanol on a rotary evaporator. NH₄PF₆ (80 mg, 0.49 mmol) was then added and the solution was placed in the freezer overnight. A black powder precipitated out and this was filtered off and washed with ether. The product was dried overnight in vacuo. Yield: 50 mg (84.7%). ¹H NMR (DMSO-d₆) δ 9.49 (d, 1H), 8.55 (d, 1H), 8.37, (t, 1H), 8.12 (d, 2H), 7.80 (t, 1H), 6.99 (d, 2H), 6.40 (d, 1H), 6.16 (t, 2H), 6.06 (d, 1H), 2.37 (septet, 1H), 2.23 (s, 3H), 0.88 (d of d, 6H).

### [Ru(biphenyl)(4-phenol-azo pyridine)Cl]PF₆ (12)

The dimer [RuCl₂(biphenyl)]₂ (30 mg, 0.05 mmol) was dissolved in a solution of 40 ml methanol and 10 ml water. The solution was refluxed under argon for 2 hours. 4-Phenol-azo pyridine (2) (20 mg, 0.1 mmol) dissolved in 4 ml methanol and 1 ml H₂O was added drop-wise and the solution immediately turned from brown to deep brown/red with a yellow tinge. The mixture was hot filtered and left to cool to room temperature whilst stirring. After thirty minutes, the volume of solvent was reduced to about 15 ml by removal of methanol on a rotary evaporator. NH₄PF₆ (84 mg, 0.5 mmol) was then added and the solution was left in the fridge overnight. The brown microcrystalline crystalline product precipitated out and was filtered off and washed with ether. The product was dried overnight in vacuo. Yield: 45 mg (46%) ¹H NMR (DMSO-d₆) δ 9.41 (d, 1H), 8.63 (d, 1H), 8.36 (t, 1H), 7.99 (d, 2H), 7.74 (t, 1H), 7.63 (d, 2H), 7.54 (t, 1H), 7.46 (t, 2H), 6.90 (d, 2H), 6.79 (d, 2H), 6.78 (d, 2H), 6.57 (t, 1H), 6.49 (t, 1H), 6.30 (t, 1H)

### [Ru(biphenyl)(4-phenol-azo pyridine)I]PF₆ (13)

The dimer [RuI₂(biphenyl)]₂ (100 mg, 0.1 mmol) was dissolved in 80 ml 75% methanol and heated to reflux for two hours. 4-Pheol-azo pyridine (2) (39.2 mg, 0.2 mmol) dissolved in 20 ml methanol was added drop-wise and the solution immediately turned from brown to intense brown yellow. The solution refluxed for a further hour, hot filtered and then the volume of solvent was reduced to about 15 ml by removal of methanol on a rotary evaporator. NH₄PF₆ (160 mg, 1 mmol) was then added and the solution was placed in the fridge overnight. A black powder product precipitated out and this was filtered off and washed with cold ethanol then ether. Yield 56.8 mg (39.1%). ¹H NMR (DMSO-d₆) δ 9.30 (d, 1H), 8.3 (d, 1H), 8.27 (t, 1H), 7.95 (d, 2H), 7.60 (t, 1H), 7.52-7.45 (m, 3H), 7.41-7.30 (m, 2H), 6.84 (t, 3H), 6.69 (t, 1H), 6.63-6.51 (m, 3H). ESI-MS m/z 582.1 (M⁺).

### [Ru(p-cymene)(3(5)-(4-dimethylaminophenylazo)pyrazole)Cl]PF₆ (14)

The dimer [RuCl₂(p-cymene)]₂ (103 mg, 0.17 mmol) was dissolved in 30 ml methanol and left to stir at room temperature until the solution turned clear. 3(5)-(4-Dimethylaminophenylazo)pyrazole (5) (69 mg, 0.32 mmol) dissolved in 10 ml methanol was added drop-wise and the solution immediately turned from brown to deep purple. The solution was stirred at room temperature for one hour. The volume of solvent was reduced to about 10 ml by removal of methanol on a rotary evaporator. NH₄PF6 (103 mg, 0.63 mmol) was then added and the solution was placed in the freezer overnight. A black powder precipitated out and this was filtered off and washed with ether. The product was dried overnight in vacuo. Yield: 126 mg (62.4%). ¹H NMR (CDCl₃) δ 8.02 (d, 2H), 7.95 (d, 1H), 7.07 (d, 1H), 6.77 (d, 2H), 6.34 (d of d, 2H), 5.68 (d of d, 2H), 3.22 (s, 6H), 2.4-2.33 (m, 4H), 0.92 (d of d, 6H).

### [Ru(biphenyl)(3(5)-(4-dimethylaminophenylazo)pyrazole)Cl]PF₆ (15)

The dimer [RuCl₂(biphenyl)]₂ (100 mg, 0.17 mmol) was dissolved in a solution of 40 ml methanol and 10 ml water. The solution was refluxed under argon for 2 hours and was hot-filtered to remove a small amount of black residue. 3(5)-(4-Dimethylaminophenylazo)pyrazole (5) (74 mg, 0.35 mmol) dissolved in 10 ml methanol was added drop-wise and the solution immediately turned from orange/brown to deep purple. The solution was stirred and left to cool to room temperature for three hours. The volume of solvent was reduced to about 20 ml by removal of methanol on a rotary evaporator. NH₄PF₆ (134 mg, 82 mmol) was then added and the solution was left in the fridge overnight during which time a dark powder precipitated out and the solution had turned to green. This product was filtered off and washed with ether. The product was dried overnight in vacuo. Yield: 153 mg (67.15%) (Found: C, 42.97; H, 3.50; N, 11.70. Calc for RuC₂₃H₂₃N₅ClPF₆: C, 42.36; H, 3.83; N, 11.67). ¹H NMR (acetone-d₆) δ 8.21 (d, 1H), 8.04 (d, 2H), 7.71-7.34 (m, 5H), 7.29 (d, 1H), 6.81 (d, 2H), 6.71 (d, 1H), 6.66-6.55 (m, 2H), 6.52 (t, 1H), 6.31 (t, 1H), 3.25 (s, 6H).

### [Ru(biphenyl)(3(5)-(4-dimethylaminophenylazo)pyrazole)I]PF₆ (16)

The dimer [RuI₂(biphenyl)]₂ (100 mg, 0.1 mmol) was dissolved in 80 ml 75% methanol and heated to reflux for three hours. 3(5)-(4-Dimethylaminophenylazo)pyrazole (5) (42.4 mg, 0.2 mmol) dissolved in 20 ml methanol was added drop-wise and the solution immediately turned from brown to dark purple. The solution refluxed for a further hour, hot filtered and then the volume of solvent was reduced to about 20 ml by removal of methanol on a rotary evaporator. NH₄PF₆ (160 mg, 1 mmol) was then added and the solution was placed in the fridge for one hour. A brown powder product precipitated out and this was filtered off and washed with cold ethanol then ether. Yield 134 mg (90.1%). %). ¹H NMR (DMSO-d₆) δ 8.07 (s, 1H), 7.87 (d, 2H), 7.47-7.32 (m, 5H), 7.25 (s, 1H), 6.73 (d, 2H), 6.64 (s, 1H), 6.47-6.30 (m, 4H), 3.14 (s, 6H). ESI-MS m/z 598.1 (M⁺).

### [Ru(benzene)(3(5)-(4-dimethylaminophenylazo)pyrazole)Cl]PF₆ (17)

The dimer [RuCl₂(benzene)]₂ (50 mg, 0.1 mmol) was dissolved in 30 ml methanol and left to stir at room temperature until the solution turned clear. 3(5)-(4-Dimethylaminophenylazo)pyrazole (5) (42.3 mg, 0.2 mmol) dissolved in 15 ml methanol was added drop-wise and the solution immediately turned from brown to deep purple. The solution was stirred at room temperature for two hours. The volume of solvent was reduced to about 10 ml by removal of methanol on a rotary evaporator. NH₄PF₆ (117 mg, 0.7 mmol) was then added and the solution was left in the freezer overnight. The volume was then reduced to around 10 ml and a black powder precipitated out. This was filtered off and washed with ether. The product was dried overnight in vacuo. Yield: 92 mg (80.00%). ¹H NMR (acetone-d₆) δ 8.31 (d, 1H), 8.21 (d, 2H), 7.31 (d, 1H), 6.96 (d, 2H), 6.29 (s, 6H).

### [Ru(THN)(3(5)-(4-dimethylaminophenylazo)pyrazole)Cl]PF₆ (18)

The dimer [RuCl₂(THN)]₂ (30 mg, 0.049 mmol) was dissolved in 15 ml methanol and left to stir at room temperature until the solution turned clear. 3(5)-(4-Dimethylaminophenylazo)pyrazole (5) (21 mg, 0.098 mmol) dissolved in 5 ml methanol was added drop-wise and the solution immediately turned from orange to deep purple. The solution was stirred at room temperature for one hour. The volume of solvent was reduced to about half by removal of methanol on a rotary evaporator. NH₄PF₆ (166 mg, 1.020 mmol) was then added and the solution was placed in the freezer overnight. A black powder precipitated out and this was filtered off and washed with ether. The product was dried overnight in vacuo. Yield: 46 mg (74.62%). ¹H NMR (CDCl₃) δ 8.15 (m, 3H), 7.21 (d, 1H), 6.93 (d, 2H), 6.35 (d, 1H), 6.0-5.8 (m, 3H), 3.0-1.5 (m, 8H).

### [Ru(THN)(4-phenol-azo pyridine)Cl]PF₆ (19)

The dimer [RuCl₂(THN)]₂(30.2 mg, 0.05 mmol) was dissolved in 15 ml methanol and left to stir at room temperature until the solution turned clear. 4-Phenol-azo pyridine (2) (21.4 mg, 0.11 mmol) dissolved in 10 ml methanol was added drop-wise and the solution turned from orange to deep brown/red with a yellow tinge. The solution was stirred at room temperature for two hours. The volume of solvent was reduced about 5 ml by removal of methanol on a rotary evaporator. NH₄PF₆ (40 mg, 0.25 mmol) was then added and the solution was placed in the freezer overnight. A black powder precipitated out and this was filtered off and washed with ether. The product was dried overnight in vacuo. Yield: 45 mg (73.41%). Found: C, 40.79; H, 3.19; N, 6.78. Calc for RuC₂₁H₂₁N₃ClOPF₆: C, 41.15; H, 3.45; N, 6.86. ¹H NMR (DMSO-d₆) δ 9.49 (d, 1H), 8.71 (d, 1H), 8.45 (t, 1H), 8.16 (d, 2H), 7.94 (t, 1H), 7.08 (d, 2H), 6.39 (d, 1H), 6.25 (t, 1H), 6.095 (t, 1H), 6.06 (d, 1H), 2.71-2.62 (m, 1H), 2.62-2.5 (m, 1H), 2.34-2.25 (m, 1H), 2.15-2.06 (m, 1H), 1.62-1.49 (m, 2H), 1.33-1.11 (m, 2H).

### [Ru(p-cymene)(Azpy-NMe₂NO₂)I]PF₆ (20)

The dimer[RuI₂(p-cymene)]₂ (12.5 mg, 0.025 mmol) was dissolved in 10 ml methanol and heated to ca. 313 K until the solution turned clear. N,N-Dimethyl-4-(2-pyridylazo)-1-nitro-aniline (6) (13.4 mg, 0.05 mmol) dissolved in 5 ml methanol was added drop-wise and the solution immediately turned from brown to bright pink. The solution was stirred at room temperature for three hours, then refluxed for 1 hour. The solution was cooled to room temperature, filtered, and the volume of solvent was reduced to about 5 ml by removal of methanol on a rotary evaporator. NH₄PF₆ (40 mg, 0.25 mmol) was then added and the solution was placed in the freezer overnight. A black microcrystalline product precipitated out and this was filtered off and washed with ether. Yield: 17.6 mg. ¹H NMR (DMSO-d₆) δ 9.66 (s, 1H), 8.27 (d, 2H), 8.46-8.36 (m, 2H), 7.88 (t, 1H), 7.47 (d, 1H), 6.32 (s, 6H), 3.17 (s, 6H).

### [Ru(p-cymene)(4-phenol-azo pyridine)I]PF₆ (21)

The dimer [RuI₂(p-cymene)]₂ (100 mg, 0.1 mmol) was dissolved in 50 ml methanol and gently heated to ca. 40° C. until the solution turned clear. 4-Phenol-azo pyridine (2) (40.8 mg, 0.2 mmol) dissolved in 10 ml methanol was added drop-wise and the solution gradually turned from brown to intense brown/yellow. The solution was cooled to room temperature, stirred for three hours, filtered and the volume reduced to ca. 10 ml on a rotary evaporator. NH₄PF₆ (160 mg, 0.1 mmol) was then added and the solution was placed in the freezer overnight. After filtration black microcrystals of the product were obtained. Yield 12 mg. ¹H NMR (DMSO-d₆) δ 9.87 (d, 1H), 8.92 (d, 1H), 8.78-8.65 (m, 3H), 8.07 (t, 1H), 7.37 (d, 2H), 6.83 (d, 1H), 6.64-6.51 (m, 3H), 3.18 (septet, 1H), 3.11 (s, 3H), 1.53 (dd, 6H). ESI-MS m/z 562.2 (M⁺).

### [(η⁶-C₆H₅OCH₂CH₂OH)Ru(azpy-NMe₂)I]I (22)

The dimer [(η⁶-C₆H₅OCH₂CH₂OH)RuI₂]₂ (121 mg, 0.12 mmol) was dissolved in 50 ml methanol and left to stir for 30 minutes. Azpy-NMe₂ (54 mg, 0.24 mmol) dissolved in methanol (20 ml) was added dropwise and the solution immediately turned deep red. The solution gradually turned to purple then to blue. The mixture was stirred at room temperature for 3 hours. The solution was filtered and the volume was reduced to 5 ml by removal of methanol on a rotary evaporator. The solution was placed in the freezer for 1 hour and the resulting bronze precipitate was filtered and washed with diethyl ether. The product was dried overnight in vacuo. Yield 93.9 mg. ¹H NMR (DMSO-d₆) δ 9.45 (d, 1H), 8.45 (d, 1H), 8.20 (d, 2H), 7.60 (t, 1H), 6.95 (d, 2H), 6.35 (t, 1H), 6.25 (t, 1H), 6.20 (d, 1H), 6.05 (d, 1H), 5.0 (t, 1H), 4.15-3.95 (m, 2H), 3.65 (m, 2H), 3.25 (s, 6H).

### [Cl(η⁶-p-cym)RuL_{B}Ru(η⁶-p-cym)Cl](PF₆)₂ (23)

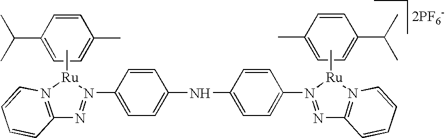

The appropriate dinucleating bizazopyridine ligand (25 mg, 0.065 mmol) was dissolved in methanol (10 mL) was added dropwise to a solution of the ruthenium dimer [(η⁶-p-cymene)RuCl₂]₂ (40 mg, 0.065 mmol) in methanol (20 mL). The solution immediately changed colour from red to blue while stirring at room temperature in an argon atmosphere shielded from light. After stirring for 2 hours, the volume of solvent was reduced and NH₄PF₆ (107 mg, 0.65 mol) was added. The blue microcrystalline precipitate, obtained after storage in a freezer overnight, was filtered off, washed with diethyl ether, and dried overnight in vacuo. Yield: 64 mg (78%) (Found: C, 38.24; H, 2.48; N, 8.00. Calcd for Ru₂Cl₂C₄₂H₄₅N₇P₂F₁₂: C, 41.66; H, 3.75; N, 8.10) ¹H NMR (methanol-d⁴): δ 9.45 (d, 2H), 8.65 (d, 2H), 8.45 (t, 2H), 8.35 (d, 4H), 7.85 (t, 2H), 7.65 (d, 4H), 6.30 (d, 4H), 6.10 (m, 4H), 2.55 (sept, 2H), 2.35 (s, 6H), 1.00 (m, 12H). ESI MS: Calcd for Ru₂C₄₂H₄₅N₇Cl₂²⁺ [M²⁺] m/z 460.3, found 459.7.

Analysis of Compounds

Ultraviolet and Visible (UV-Vis) Spectroscopy

A Perkin-Elmer Lambda-16 UV-Vis spectrophotometer was used with 1-cm path-length quartz cuvettes (0.5 mL) and a PTP1 Peltier temperature controller. Spectra were recorded at 25° C. for aqueous solutions from 800-200 nm. Spectra were processed using UVWinlab software for Windows 95.

Aqueous Solution Chemistry:

The complex was dissolved in H₂O/D₂O, the pH was taken, and NMR spectra were recorded at uniform time intervals at a fixed temperature using a multi-zg kinetic experiment program. After acquisition, Electrospray Mass Spectrometry was performed on a portion of the NMR solution.

Cytotoxicity Studies

Compounds were tested for inhibitory growth activity against the A2780 and A549 cancer cell lines. Each drug was tested for activity at six different concentrations (100 μM, 50 μM, 10 μM, 5 μM, 1 μM and 0.1 μM) and each concentration was tested in triplicate, relative to a cisplatin control.

The A2780 cancer cell line was maintained by growing the cells in RPMI media supplemented with 5% fetal bovine serum, 1% penicillin/streptomycin and 2 mM L-glutamine. The cells were split when approximately 70-80% confluence were reached using 0.25% trypsin/EDTA. The cells were kept incubated at 37° C., 5% CO₂, high humidity. The A549 cancer cell line was maintained by growing the cells in DMEM media supplemented with 10% fetal bovine serum, 1% penicillin/streptomycin and 2 mM L-glutamine. The cells were split when approximately 70-80% confluence were reached using 0.25% trypsin/EDTA. The cells were kept incubated at 37° C., 5% CO₂, high humidity.

A2780 cancer cells were plated out at 50000 cells/well (±10%) on day one. A549 cancer cells were plated out at 20000 cells/well (±10%) on day two. On day three the test compound was dissolved in DMSO to give a stock solution of 20 mM and serial dilutions were carried out in DMSO to give concentrations of drug in DMSO of 10 mM, 2 mM, 1 mM, 0.2 mM and 0.02 mM. These were added to the wells to give the six testing concentrations and a final concentration of DMSO as 0.5% (v/v) with a total volume of drugs and media to be 200 μl. The cells were exposed to the drug for 24 hours then, after drug removal, fresh media was given and the cells were incubated for 96 hours recovery time. The remaining biomass was estimated by the sulforhodamine B assay. The cells were then fixed using 50 μl 50% (w/v) TCA and incubated at 4° C. for one hour. The biomass was stained with 100 μl 0.4% (w/v) sulforhodamine B in 1% acetic acid. The dye was solubilised with Tris Buffer and the absorbance was read using a BMG Fluostar microplate reader at 595 nm. A baseline correction at 690 nm was subtracted from the values. The absorbance for 100% cell survival was based on the average absorbance for the 0.1 μM dosed triplicate for that drug. IC₅₀ values were calculated using XL-Fit version 4.0.

Results

Ultraviolet and Visible (UV-Vis) Spectroscopy

TABLE 1

| Compound | λmax/nm | ε/M⁻¹cm⁻¹ | Assignment |
|---|---|---|---|
| 1 | 318, 445 | 18000, 420 | π→π*, n→π* |
| 2 | 246, 358 | 10000, 25000 | π→π*,, π→π* |
| 5 | 267, 402 | 12000, 33000 | π→π*, π→π* |
| 9 | 263, 460, 628 | 22000, 12000, 65000 | π→π*, π→π*, Ru 4d⁶→π* |
| 11 | 266, 434, 573 | 10000, 16000, 11000 | π→π*, π→π*, Ru 4d⁶→π* |
| 12 | 267, 436, 586 | 20000, 18000, 12000 | π→π*, π→π*, Ru 4d⁶→π* |
| 14 | 302, 469, 577 | 9000, 14000, 22000 | π→π*, π→π*, Ru 4d⁶→π* |
| 15 | 287, 481, 592 | 13000, 13000, 26000 | π→π*, π→π*, Ru 4d⁶→π* |
| 17 | 303, 469, 573 | 8000, 13000, 21000 | π→π*, π→π*, Ru 4d⁶→π* |
| 18 | 303, 481, 581 | 8000, 12000, 23000 | π→π*, π→π*, Ru 4d⁶→π* |
| 19 | 266, 432, 573 | 12000, 19000, 10000 | π→π*, π→π*, Ru 4d⁶→π* |

The solution chemistry was followed by ¹H NMR unless stated otherwise.

Aqueous Solution Chemistry

### [Ru(p-bip)(2-phenyl-azo pyridine)I]PF₆ (7)

Conditions: 100 μM, 95% D₂O, 5% MeOD, pH=7, 37° C., Over 24 Hours

This experiment was designed to mimic the cell testing conditions of complex 7. No hydrolysis occurred over the 24 hours, although there was a small (about 5%) loss of the arene from the complex.

### [Ru(p-cymene)(Azpy-NMe₂)I]PF₆ (8)

Conditions: Saturated Solution (Filtered), 90% H₂O/10% D₂O, pH=7.2, 25° C. Over 24 Hours

No change occurred over 24 hours at 25° C. indicating that the complex stays as the intact ruthenium(II) arene iodide complex.

Conditions: 100 μM, 99.5% D₂O, 0.5% DMSO, 115 mM NaCl, pH=7.5, 37° C., Over 24 Hours

This experiment was designed to mimic the cell testing conditions of complex 8. No change occurred over the 24 hours, indicating that the complex stays as the intact ruthenium(II) arene iodide complex.

### [Ru(biphenyl)(Azpy-NMe₂)Cl]PF₆ (9)

Conditions: Saturated Solution (Filtered) in 100% D₂O, 25° C. Over 25 Hours

The spectrum was recorded every 2 hours over a 25 hour time period. No changes occurred in the spectrum over time.

Conditions: 100 μM, 99.5% D₂O, 0.5% DMSO, 115 mM NaCl, 37° C. Over 24 Hours

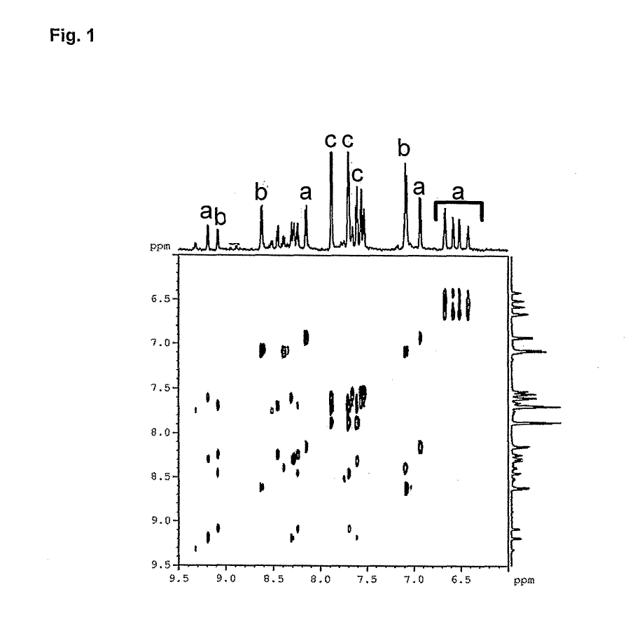

This experiment was designed to mimic the cell testing conditions of the complex. Any possible hydrolysis is suppressed by the high salt content and arene loss had occurred for ca. 40% of the solution after 24 hours, about 50% of the solution exists as the intact chloro complex. The ¹H 2D TOCSY of complex 9 after 24 hours is shown in FIG. 1, where species a is the intact chloride complex, species b is the environment for the ligand after arene loss and species c is free biphenyl.

Conditions: 90% H₂O/10% D₂O to Give a Concentration of ca. 100 μM, Initial pH 6.42 at 310 K.

Spectra were recorded every hour over 24 h. After 24 h the speciation was 24% intact cation, 9% hydrolysis, 67% arene loss. The decay of the intact cation in solution appeared to follow pseudo first kinetics to give a half life of decomposition of 20.27 hours.

### [Ru(biphenyl)(Azpy-NMe₂)I]PF₆ (10)

Conditions: Saturated Solution, 90% H₂O/10% D₂O, 37° C. Over 24 Hours

No change occurred over 24 hours at 37° C. indicating that the complex stays as the intact ruthenium(II) arene iodide complex.

### [Ru(p-cymene)(4-phenol-azo pyridine)Cl]PF₆ (11)

Conditions: 100 μM, 90% H₂O/10% D₂O, pH=ca 5.8, 25° C. & 37° C., Over 24 Hours

At 25° C. after 24 hours, SD010 existed as 69% in the initial chloro form (confirmed by MS m/z=470.12, calcd m/z=470.06), 10% as the hydrolysed product (confirmed by the addition of excess NaCl to give 100 mM) and 20% as the complex after arene loss. At 37° C. after 24 hours, only 32% exists as the initial chloro complex, 31% has hydrolysed and 36% is minus arene. The decay of the intact cation in solution at 310 K appeared to follow pseudo first kinetics to give a half life of decomposition of 21.03 h.

### [Ru(biphenyl)(4-phenol-azo pyridine)Cl]PF₆ (12)

Conditions: 90% H₂O/10% D₂O to Give a Concentration of ca. 100 μM, Initial pH 5.46 at 310 K.

Spectra were recorded every hour over 24 h. After 24 h the speciation was 31% intact cation, 5% hydrolysis, 64% arene loss. The decay of the intact cation in solution appeared to follow pseudo first kinetics to give a half life of decomposition of 13.05 hours.

### [Ru(biphenyl)(4-phenol-azo pyridine)I]PF₆ (13)

Conditions: Saturated Solution, 90% H₂O/10% D₂O, 37° C. Over 24 Hours

No change occurred over 24 hours at 37° C. indicating that the complex stays as the intact ruthenium(II) arene iodide complex.

Conditions, 50 μM, 95% H₂O, 5% MeOH, pH 2.25.

The change in absorbance at 620 nm was followed by time by UV-Vis spectroscopy. The decay appeared to follow pseudo first order kinetics to give a half live for hydrolysis of 2.14 h.

### [Ru(p-cymene)(3(5)-(4-dimethylaminophenylazo)pyrazole)Cl]PF₆ (14)

Conditions: 100 μM, 90% H₂O/10% D₂O, pH=ca 5, 25° C. & 37° C. Over 24 Hours

Initially (time=40 mins) at 25° C. there are two sets of peaks corresponding to chloride species and aqua species (approximate ratio 60%:40%). After 24 hours the complex exists fully in the aqua form (MS was performed on the solution and no peak corresponding to the intact chloride species was detected). The presence of the aqua species was confirmed by adding excess NaCl to give 100 mM solution and observing the peaks due to aqua decreasing and the peaks due to the chloride species emerging. At 37° C. the course of the reaction is the same.

Conditions: 50 μM, 95% H2O, 5% MeOH, pH 2.27.

The change in absorbance at 620 nm was followed by time by UV-Vis spectroscopy. The decay appeared to follow pseudo first order kinetics to give a half live for hydrolysis of 2.09 h.

### [Ru(biphenyl)(3(5)-(4-dimethylaminophenylazo)pyrazole)Cl]PF₆ (15)

Conditions: 100 μM, 99.5% D₂O, 0.5% DMSO, 115 mM NaCl, pH=7.35, 37° C., Over 24 Hours

This experiment was designed to mimic the cell testing conditions of the complex. The pH was initially 6.4 but was adjusted to 7.35 before incubation at 37° C. Any possible hydrolysis is suppressed by the high salt content. There are major peaks corresponding to free biphenyl.

### [Ru(benzene)(3(5)-(4-dimethylaminophenylazo)pyrazole)Cl]PF₆ (17)

Conditions: Saturated Solution, 90% H₂O/10% D₂O, pH=ca. 4.5, 25° C. Over 24 Hours

Initially (time=45 mins), ca. 77% of the intact chloride complex remained. After 24 hours about 60% of complex 17 exists as the intact chloride complex and 40% has hydrolysed (confirmed by adding (undefined) excess NaCl and watching the aqua peak disappear).

Conditions, 50 μM, 95% H₂O, 5% MeOH, pH 2.30.

The change in absorbance at 620 nm was followed by time by UV-Vis spectroscopy. The decay appeared to follow pseudo first order kinetics to give a half live for hydrolysis of 2.67 h.

### [Ru(THN)(4-phenol-azo pyridine)Cl]PF₆ (19)

Conditions: Saturated Solution, 90% H₂O/10% D₂O, pH=ca.5.8, 25° C., Over 24 Hours

After 24 hours at 25° C. the solution showed two sets of peaks, intact chloride species and hydrolyzed species (accounting for 87% and 13% respectively). The hydrolysis was confirmed by adding excess (undefined) NaCl and observing the peaks assigned to the aqua complex disappear.

### [Ru(p-cymene)(4-phenol-azo pyridine)I]PF6 (21)

Conditions: 100 μM, 90% H₂O/10% D₂O, pH=ca. 6.5, 37° C. Over 24 Hours

No change occurred over 24 hours at 37° C. indicating that the complex stays as the intact ruthenium(II) arene iodide complex.

Cytotoxicity

| Compound | A2780 IC₅₀ (μM) | A549 IC₅₀ (μM) |
|---|---|---|
| 7 | 51 | 39 |
| 8 | 5 | 3 |
| 9 | 40 | 53 |
| 10 | 3 | 2 |
| 11 | 54 | — |
| 12 | 18 | 56 |
| 13 | 5 | 6 |
| 14 | 18 | 41 |
| 15 | 24 | 32 |
| 16 | 31 | 42 |
| 17 | 88 | — |
| 18 | 57 | — |
| 19 | 38 | 81 |
| 20 | 40 | 28 |
| 21 | 4 | 4 |
| 22 | 15 | 49 |
| 23 | 80 | — |

Further Analysis of Compounds

Solution Chemistry

Solutions of four ruthenium complexes (8, 10, 13 and 21) in MeOD were diluted down in 10 mM phosphate buffer/D₂O to give a final concentration of 100 μM ruthenium (95% D₂O, 5% MeOD) and NMR spectra were recorded at 310 K initially (time ca. 15 minutes) and after 24 h. The pH* of the samples was 7.35 (8), 7.40 (10), 7.31 (13) and 7.38 (21), The samples were kept in the water bath at 310 K between acquisitions. After 24 hour, ESI-MS was performed on the samples. These conditions were chosen to mimic pH, concentration, exposure time and temperature for the biological cell tests. No new peaks/peak shifts occurred in the spectra over 24 hours suggesting that no hydrolysis had occurred; this hypothesis was confirmed by performing ESI-MS on the NMR solutions where only one mass corresponding to the intact cation was observed (8 m/Z 588.75 (M+), 10 m/Z 608.71 (M+), 13 m/Z 581.65 (M+), 21 m/Z 561.70 (M+).

Electrochemistry and Cyclic Voltammetry

Electrochemical studies were performed with General Purpose Electrochemical System (GPES) Version 4.5 software connected to an Autolab system containing a PSTAT20 potentiostat. All of the electrochemical techniques used a three-electrode configuration. The reference electrode used was Ag/AgCl in a solution of 0.1 M [TBA][BF₄] in DMF against which [symbol] for the ferrocinium/ferrocene couple was measured to be +0.55 V. The working and counter electrodes were a platinum microdisc (0.5 mm diameter) and a large surface area platinum wire respectively. Coulometric experiments were performed in a conventional H-type cell using large surface-area Pt working and counter electrodes. All solutions were purged with dry nitrogen prior to electrochemical study. The electrochemical reductions of all six ruthenium complexes were studied by cyclic voltammetry in DMF. The main characteristics observed are as follows: All complexes displayed two electrochemical reductions, the first occurred at ca. −0.2 to −0.4V (13−0.26 V, 21−0.33 V, 10−0.36 V, 8−0.40 V) and a second near −2 V, which was not characterized further due to being close to the solvent cut off and being considered as biologically inaccessible anyway. Complexes are reduced at a more positive potential as the arene is changed from p-cymene to biphenyl and as the chelating azo ligand is changed from azpy-NMe₂ to azpy-OH. In the biphenyl case this first reduction is essentially irreversible (no return peak observed) and the complex undergoes an EC type (Electrochemical-Chemical) reaction where a new peak appears on the return sweep that is the re-oxidized ‘daughter’ product. The same type of EC type reaction occurs for the p-cymene complexes except there is some degree of reversibility (quasi-reversible) for the initial reduction reaction. These results show that the complexes can be electrochemically reduced at biologically relevant potentials.

Reactions with Ascorbate

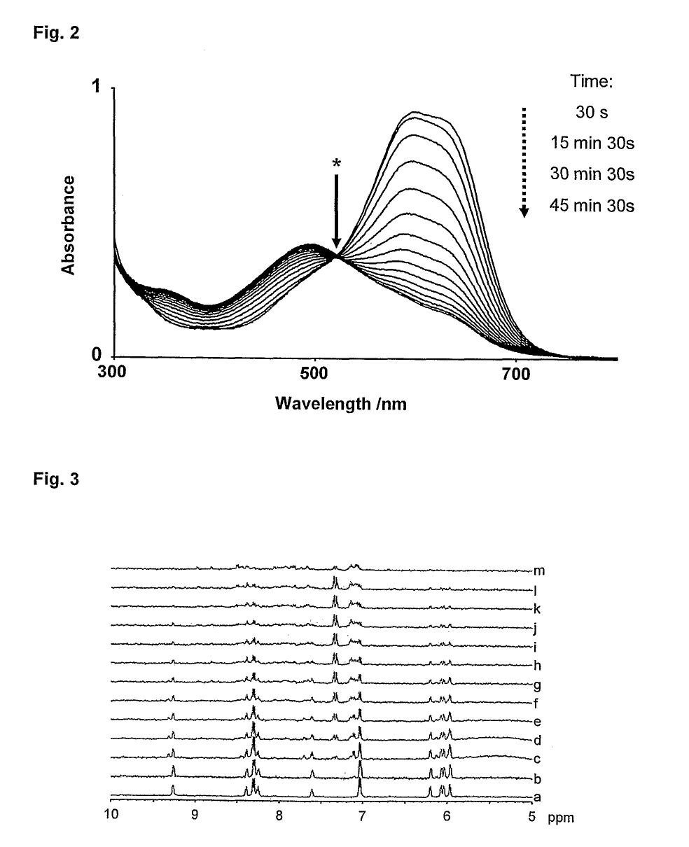

Initially the reaction of complex 8 and 5 equivalents ascorbate in 10 mM phosphate buffer solution (pH 7.35) at 310K was investigated by UV-Vis spectroscopy over 4 hours where the decrease in intensity of the Ruthenium-phenylazopyridine MLCT band and the presence of an isobestic point (at ca. 520 nm) suggested a single step reaction from starting material to reduced product (FIG. 2). The same reaction was followed by ¹H NMR in 10 mM phosphate buffer/D₂O (pH 7.30) and the disappearance of all proton peaks suggested that a one electron reduction was occurring (i.e. going from a diamagnetic NMR active species to a paramagnetic NMR inactive species, FIG. 3—a: 8; b: 8+5 eq. ascorbate after 5 minutes 47 seconds; c-l: every 30 minutes thereafter; m: after 24 hours). Complexes 10, 13 and 21 were similarly reduced. These results show that biological reductants can reduce compounds of the present invention.

Detection of Reactive Oxygen Species (ROS) in A549 Cancer Cells

The generation of ROS can be detected inside living cells using the molecular probe DCFH-DA. This probe crosses the membrane into cells where it is hydrolyzed to DCFH. In the presence of ROS it is oxidized to highly fluorescent DCF. A549 cancer cells were plated out at a density of 20000 cells per well into black 96 well plates and were incubated at 310 K, 5% CO₂, high humidity for 24 hours. Cells were loaded with DCFH-DA (10 μM, 0.5% DMSO (v/v)) and were incubated at 310 K, 5% CO₂, high humidity for 30 minutes. The probe was removed and the cells were washed twice with PBS (200 μL). The cells were then kept in Hanks Balanced Salt Solution (HBSS) and the ruthenium compounds were diluted with HBSS and added to the wells (25 μM, 0.5% DMSO (v/v)). Hydrogen peroxide (25 μM) was added as a positive control and the fluorescence was read every 200 seconds over a period of 6.5 hours at 310 K by excitation at 480±10 nm and emission at 538±15 nm on a BMG fluostar plate reader. A time course experiment was performed to follow any increase in fluorescence over 6.5 hours after addition of ruthenium compounds to cells pre-loaded with DFCH-DA. This allowed evaluation of the generation (if any) of ROS due to any reduction of ruthenium inside cells, Compounds chosen for this study were 8, 10, 13 and 21 as well as RM175, a compounds which is thought to exert its cytotoxic effect from binding to DNA and not through ROS generation.

| Compound | Structure | Reference |
|---|---|---|
| RM175 | 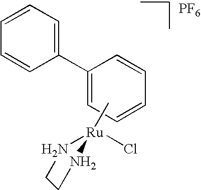 | Example 9, WO 2001/030790 |

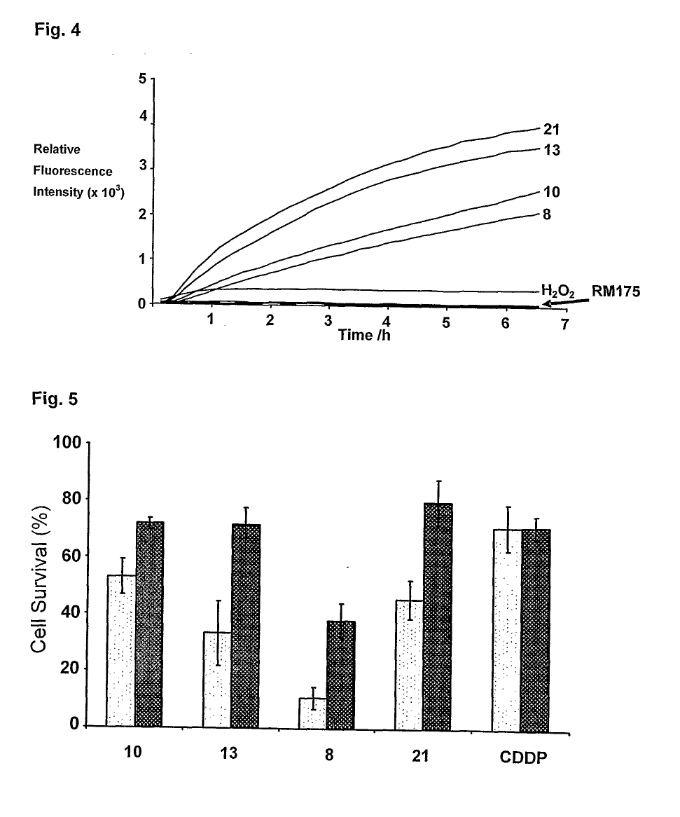

FIG. 4 shows the increase in fluorescence detected over time. Compounds 8, 10, 13 and 21 all cause an increase in the DCF fluorescence detected with time, and to a much greater extent than the hydrogen peroxide control. This indicates that these compounds generate ROS inside A549 cancer cells. In contrast, RM175 did not cause an increase in DCF fluorescence above the baseline value, which shows that this compound does not generate ROS.

Cell Viability After Increasing Thiol Levels

Cell viability was determined in the A549 cancer cell line after cells were pre-incubated with 5 mM NAC to increase intracellular thiol levels. FIG. 5 shows the cell viability for the four ruthenium compounds after 24 hours incubation with the drug (1 μM—10; 5 μM—8, 13, 21; 5 μM—CDDP control) and 96 hour recovery time at selected concentrations for both the untreated cells (lighter bars)and cells pre-treated with 5 mM NAC for two hours (darker bars). In all cases there is a greater cell survival for the cells that have increased thiol levels. This implies that ROS are involved in cell death.

1. A ruthenium (II) compound of formula (I): 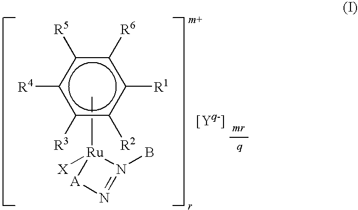
or a solvate or prodrug thereof, wherein:
R¹, R², R³, R⁴, R⁵ and R⁶ are independently selected from H, C₁₋₇ alkyl, C₅₋₂₀aryl, C₃₋₂₀ heterocyclyl, halo, ester, amido, acyl, sulfo, sulfonamido, ether, thioether, azo, amino, or R¹ and R² together with the ring to which they are attached form a saturated or unsaturated carbocyclic or heterocyclic group containing up to three 3- to 8-membered carbocyclic or heterocyclic rings, wherein each carbocyclic or heterocyclic ring may be fused to one or more other carbocyclic or heterocyclic rings;
X is halo or a neutral or negatively charged O, N- or S-donor ligand;
Y is a counterion;
m is 0 or 1;
q is 1, 2 or 3;
A is either:
(i) ^{(Ru)}—NR^{N1}R^{N2}—R^{N3}—^{(N)}, where R^{N1} and R^{N2} are independently selected from H, optionally substituted C₁₋₇ alkyl, C₃₋₂₀ heterocyclyl and C₅₋₂₀ aryl, and R^{N3} is C₁₋₂ alkylene; or
(ii) a nitrogen-containing C₅₋₆ aromatic ring, wherein the nitrogen ring atom is bound to the ruthenium atom, and the ring is also bound to the azo-nitrogen, either by a single bond wherein the bond is α or β to the nitrogen ring atom, or by a —CH₂— group wherein the bond is α to the nitrogen ring atom;
B is optionally substituted C₁₋₇ alkyl, C₃₋₂₀ heterocyclyl or C₅₋₂₀ aryl;
the compound of formula (I) optionally being in the form of a dimer in which:
(a) the B group from each moiety are linked through a linking group which is a single bond, —O—, —NH—, C₁₋₆ alkylene or C₅₋₂₀ arylene;
(b) one group serves as the B group for both moieties; or
(c) R¹ on each moiety together form a linking group which is a single bond, —O—, C₁₋₆ alkylene or C₅₋₂₀ arylene.

2. The compound according to claim 1, wherein A is a nitrogen-containing aromatic ring, wherein the nitrogen ring atom is bound to the ruthenium atom, and the ring is further bound to the azo-nitrogen by a single bond α or β to the nitrogen ring atom or by a —CH₂— group α to the nitrogen ring atom.

3. The compound according to claim 2, wherein A is bound to the azo-nitrogen by a single bond α to the nitrogen ring atom.

4. The compound according to claim 3, wherein A is a pyridine or pyrazole ring.

5. The compound according to claim 4, wherein A is unsubstituted.

6. The compound according to claim 1, wherein B is phenyl optionally substituted with a group selected from —OR^{O1}, —NR^{N1}R^{N2}, —NO₂, —C₁₋₇ alkyl, —C₅₋₂₀) aryl, wherein R^{O1}, R^{N1} and R^{N2} are independently selected from H or C₁₋₇ alkyl.

7. The compound according to claim 6, wherein B is unsubstituted phenyl.

8. The compound according to claim 6, wherein B is para-dimethylaminophenyl or p-hydroxyphenyl.

9. The compound according to claim 1, wherein X is halo.

10. The compound according to claim 9, wherein X is chloro or iodo.

11. The compound according to claim 1, wherein R¹ and R² together with the ring to which they are attached form a saturated or unsaturated carbocyclic or heterocyclic group containing up to 3- to 8-membered carbocyclic or heterocyclic rings, wherein each carbocyclic or heterocyclic ring may be fused to one or more other carbocyclic or heterocyclic rings.

12. The compound according to claim 11, wherein R³, R⁴, R⁵ and R⁶ are H.

13. The compound according to claim 1, wherein R¹, R², R³, R⁴, R⁵ and R⁶ are independently selected from C₁₋₇ alkyl, C₅₋₂₀aryl, C₃₋₂₀ heterocyclyl, halo, ester, amido, acyl, sulfo, sulfonamido, ether, thioether, azo and amino.

14. The compound according to claim 13, wherein R¹, R², R³, R⁴, R⁵ and R⁶ are independently selected from H and C₁₋₇ alkyl.

15. The compound according to claim 14, wherein at least four of R¹, R², R³, R⁴, R⁵ and R⁶ are hydrogen.

16. A composition comprising a compound according to claim 1, and a pharmaceutically acceptable carrier or diluent.

17. (canceled)

18. A method for the preparation of a composition for the treatment of cancer comprising combining a compound according to claim 1 with a pharmaceutically acceptable carrier or diluent.

19. A method of treatment of a subject suffering from cancer, comprising administering to such a subject a therapeutically-effective amount of a compound according to claim 1.
